(19) United States
(12) Patent Application Publication
(10) Pub. No.: US 2023/0338464 A1
(43) Pub. Date: Oct. 26, 2023

(54) USE OF ANTI-AGING GLYCOPEPTIDES FOR TREATMENT OF DRY EYE DISEASE, RETINAL DEGENERATIVE DISEASES, OR OCCULAR INFLAMMATION

(71) Applicants: Lachlan Grant Young, Nanimo (CA); Protokinetix Inc., Marietta, OH (US)

(72) Inventor: Lachlan Grant YOUNG, Nanimo (CA)

(21) Appl. No.: 17/906,003

(22) PCT Filed: Mar. 10, 2021
(86) PCT No.: PCT/CA2021/050323

§ 371 Date: Sep. 9, 2022

Related U.S. Application Data
(60) Provisional application No. 62/987,522, filed on Mar. 10, 2020.
(60) Provisional application No. 63/074,222, filed on Sep. 3, 2020.
(60) Provisional application No. 63/077,749, filed on Sep. 14, 2020.
(60) Provisional application No. 63/118,712, filed on Nov. 26, 2020.

(51) Int. Cl. A61K 38/14; A61K 9/00; A61P 27/04

(52) U.S. Cl. CPC A61K 38/14; A61K 9/0048; A61P 27/04

(57) ABSTRACT

The present document describes methods of using a gem-difluorinated C-glycopeptide compound of general formula I, or a pharmaceutically acceptable base, addition salt with an acid, hydrate or solvate of the compound of general formula I for the treatment or prevention of dry eye disease in a subject in need thereof and/or for the treatment or prevention of a retinal degenerative disease, an ocular inflammation, or a combination thereof.

### CROSS-REFERENCE TO RELATED APPLICATIONS

This application claims priority of U.S. provisional patent application No. 62/987,522, filed on Mar. 10, 2020; U.S. provisional patent application No. 63/074,222, filed Sep. 3, 2020; U.S. provisional patent application No. 63/077,749, filed Sep. 14, 2020; and U.S. provisional patent application No. 63/118,712, filed Nov. 26, 2020, the specifications of which are hereby incorporated by reference in their entireties.

### BACKGROUND

### (A) Field

The subject matter disclosed generally relates to a method for the treatment or prevention of dry eye disease, and more particularly to a method for the treatment or prevention of dry eye disease in a subject in need thereof.

### (b) Related Prior Art

Antifreeze biological compounds, and particularly glycoproteins, exist in the natural environment. These compounds are present for example in some fishes, enabling them to survive in a low temperature environment (i.e. near zero or sub-zero temperatures). Scientists have been investigating how antifreeze compounds taken from the natural environment (fish, amphibians, plants, insects, etc.) have an influence on these phenomena. Research has focused on the synthesis of analogous compounds that are sufficiently stable and whose activity is at least equal to or even greater than the activity of the natural molecules, for commercial applications.

Anti-freeze proteins (AFP) have generated increasing interest for their ability to protect cells under a variety of conditions. They are naturally encountered in Arctic and Antarctic fish as well as other cold-climate dwelling invertebrates and are responsible for maintaining cells and tissue functional at sub-zero temperatures. AFP were successfully isolated in the 1950s and have demonstrated the ability to non-colligatively lower the freezing temperature of body fluids by binding to ice crystals.

Early experiments with these compounds in the field of organ and tissue transplantation showed promising results, making them attractive therapeutic candidates to protect cells against harmful conditions associated with the process of retrieval-preservation-reperfusion. Moreover, further benefits have also been demonstrated during cryopreservation of different cells, including islets of Langerhans, which significantly increased their viability and function when supplemented with AFP during cryostasis. The Anti-aging glycopeptides (AAGP™) used in the present invention derive from attempts to obtain analogs of anti-freeze glycoproteins.

Anti-aging glycopeptides (AAGP™) compounds are gem difluorinated C-glycopeptides which have been proposed to have applicability under harsh cellular stresses, such as nutrient deprivation, high temperature and cryopreservation, oxidative stress from hydrogen peroxide (H₂O₂), UV irradiation, and inflammation.

Dry eye, also known as dry eye disease or keratoconjunctivitis sicca, is a multifactorial disease of the tears and ocular surface that results in symptoms of discomfort, visual disturbance, and tear film instability with potential damage to the ocular surface. It is accompanied by increased osmolarity of the tear film and inflammation of the ocular surface (The Ocular Surface, “The Definition and Classification of Dry Eye Disease: Report of the Definition and Classification Subcommittee of the International Dry Eye Workshop (2007),” 5(2): 75-92 (2007)). Dry eye is recognized as a disturbance of the lacrimal functional unit, an integrated system comprising the lacrimal glands, ocular surface (cornea, conjunctiva and meibomian glands) and lids, and the sensory and motor nerves that connect them. The lacrimal functional unit controls the major components of the tear film in a regulated fashion and responds to environmental, endocrinological, and cortical influences. The unit's function is to preserve the integrity of the tear film, the transparency of the cornea, and the quality of the image projected onto the retina. Disease or damage to any component of the lacrimal functional unit (the afferent sensory nerves, the efferent autonomic and motor nerves and the tear-secreting glands) can destabilize the tear film and lead to ocular surface disease that expresses itself as dry eye.

The major classes of dry eye are aqueous tear-deficient dry eye (ADDE) and evaporative dry eye (EDE). ADDE is due to failure of lacrimal tear secretion and this class can be further subdivided to Sjogren syndrome dry eye (the lacrimal and salivary glands are targeted by an autoimmune process, e.g., rheumatoid arthritis) and non-Sjogren's syndrome dry eye (lacrimal dysfunction, but the systemic autoimmune features of Sjogren's syndrome are excluded, e.g., age-related dry eye). EDE is due to excessive water loss from the exposed ocular surface in the presence of normal lacrimal secretory function. Its causes can be intrinsic (due to intrinsic disease affecting lid structures or dynamics, e.g., meibomian gland dysfunction) or extrinsic (where ocular surface disease occurs due to some extrinsic exposure, e.g., vitamin A deficiency).

Dry Eye is one of the most common ocular problems with an estimated prevalence of 4.91 million people in the United States affecting around 3.23 million women and 1.68 million men over the age of fifty. Current therapies for dry eye are palliative with a focus on the replacement of tears to reduce symptoms. Over-the-counter artificial tear formulations are available. In addition, a non-pharmacological approach for improving aqueous tear film content is punctual tamponade occlusion. However, punctual tamponade occlusion carries the risk of reduced tear production, clearance and ocular surface sensation. While these palliative therapies have benefits over the short term, they have limited utility in long-term control therapy for dry eye. RESTASIS® (cyclosporine A) is the first prescription product for dry eye therapy. RESTASIS® increases tear production in patients whose tear production is suppressed as a result of ocular inflammation associated with dry eye disease.

However, there is a need for therapies that have a broader application than anti-inflammatory medication.

Therefore, a need exists in the art for alternative methods of treating dry eye, retinal degenerative diseases, or ocular inflammation.

Therefore, there exists a need in the art for means of treating or preventing dry eye disease, retinal degenerative diseases, or ocular inflammation.

### SUMMARY

According to an embodiment, there is provided a method for the treatment or prevention of dry eye disease in a subject in need thereof comprising the step of:
a) administering to an eye of the subject an effective amount of a gem-difluorinated C-glycopeptide compound of general formula I, or a pharmaceutically acceptable base, addition salt with an acid, hydrate or solvate of the compound of general formula I:

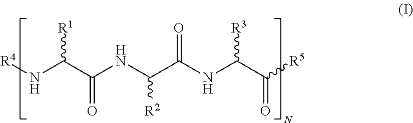

in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

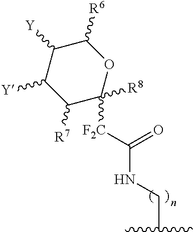

in which:
   n is an integer between 3 and 4,
   Y, Y′ are independent groups
      in which Y, Y′=H, OR, N₃, NR′R″, or SR′″,
         where R=H, benzyl, trimethylsilyl, tert-butyldimethylsilyl, tert-butyldiphenylsilyl or acetate group,
         R′, R″ independently=H, alkyl, allyl, benzyl, tosylate group, C(═O)-alkyl or C(═O)—Bn,
         R′″=H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R³ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃
      then R³=

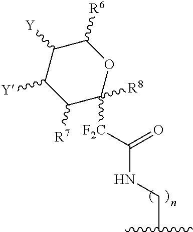

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

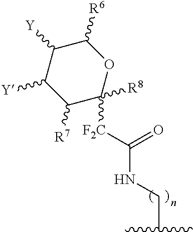

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²=R³=H, CH₃, CH(CH₃)₂, CH₂CH(CH₃)₂, or CH(CH₃)CH₂CH₃
   then R¹=

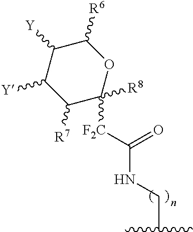

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject may be a human subject.

The compound of formula I may be a compound of formula II:

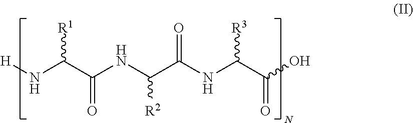

in which: N is an integer between 1 and 5,
   and
   R¹, R², R³ are independent groups in which two of R¹, R² and R³ are selected from H, CH₃ and the remaining R¹, R² and R³ is

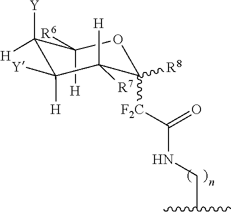

in which: n is an integer between 3 and 4,
   Y, Y′ are independent groups
      in which Y, Y′=H, OR, N₃, NR′R″ or SR′″,
         where R=H, benzyl, trimethylsilyl, tert-butyldimethylsilyl, tert-butyldiphenylsilyl, or acetate group,
         R′, R″ independently=H, alkyl, allyl, benzyl, tosylate group, C(═O)-alkyl, or C(═O)—Bn,
         R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R³ is a hydrogen atom H or a free or protected alcohol function,
      and
      if R¹=R²=H or CH₃,
      then R³=

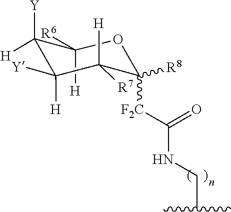

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹=R³=H or CH₃,
   then R²=

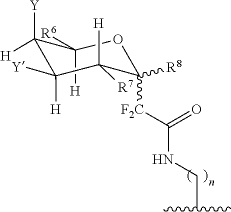

in which: n is an integer between 3 and 4,
Y, Y′ are independent groups
   in which Y, Y′=H, OR, N₃, NR′R″, SR′″,
   where R=H, benzyl, trimethylsilyl, tert-butyldimethylsilyl, tert-butyldiphenylsilyl, or acetate group,
   R′, R″ independently=H, alkyl, allyl,
   Bn, tosylate, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²=R³=H or CH₃,
   then R¹=

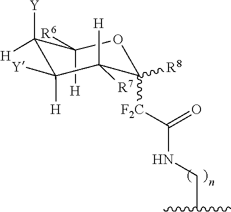

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula III:

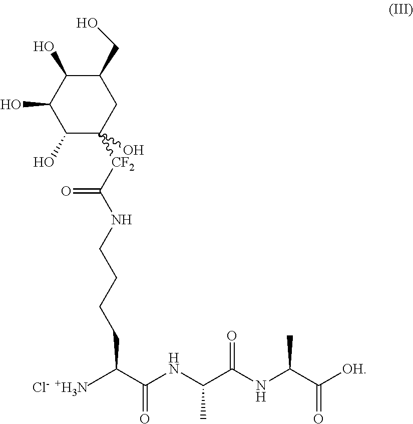

The effective amount may be with from about from about 0.01 mg/ml to about 5 mg/ml of the compound of formula I, formula II or formula III.

The effective amount may be with from about from about 1 mg/ml to about 5 mg/ml of the compound of formula I, formula II or formula III.

The administration may be at least once, twice, three times, or four times per day.

According to another embodiment, there is provided a use of a gem-difluorinated C-glycopeptide compound of general formula I, or a pharmaceutically acceptable base, addition salt with an acid, hydrate or solvate of the compound of general formula I for the treatment or prevention of dry eye disease in a subject in need thereof:

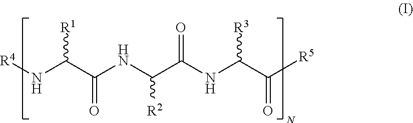

in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

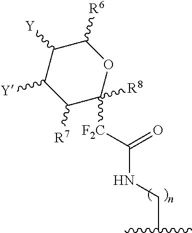

in which:
   n is an integer between 3 and 4,
   Y, Y′ are independent groups
      in which Y, Y′=H, OR, N₃, NR′R″, or SR′″,
         where R=H, benzyl, trimethylsilyl, tert-butyldimethylsilyl, tert-butyldiphenylsilyl or acetate group,
         R′, R″ independently=H, alkyl, allyl, benzyl, tosylate group, C(═O)-alkyl or C(═O)—Bn,
         R′″=H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃
      then R³=

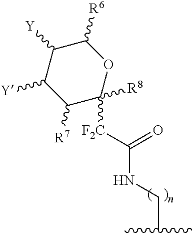

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

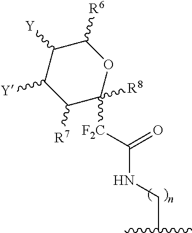

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group, R³ is a hydrogen atom H or a free or protected alcohol function,
   if R²=R³=H, CH₃, CH(CH₃)₂, CH₂CH(CH₃)₂, or CH(CH₃)CH₂CH₃
   then R¹=

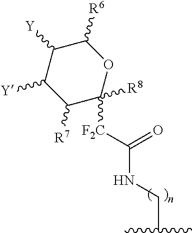

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject may be a human subject.

The compound of formula I may be a compound of formula II:

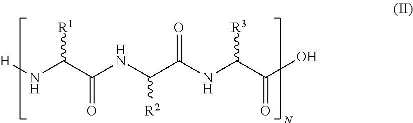

in which: N is an integer between 1 and 5,
   and
   R¹, R², R³ are independent groups in which two of R¹, R² and R³ are selected from H, CH₃ and the remaining R¹, R² and R³ is

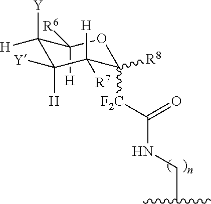

in which: n is an integer between 3 and 4,
   Y, Y′ are independent groups
      in which Y, Y′=H, OR, N₃, NR′R″ or SR′″,
         where R=H, benzyl, trimethylsilyl, tert-butyldimethylsilyl, tert-butyldiphenylsilyl, or acetate group,
         R′, R″ independently=H, alkyl, allyl, benzyl, tosylate group, C(═O)-alkyl, or C(═O)—Bn,
         R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R⁸ is a hydrogen atom H or a free or protected alcohol function,
      and
      if R¹=R²=H or CH₃,
      then R³=

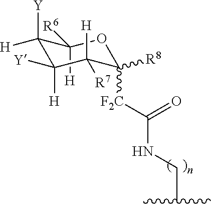

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹=R³=H or CH₃,
   then R²=

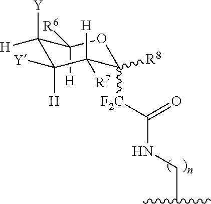

in which: n is an integer between 3 and 4,
Y, Y′ are independent groups
   in which Y, Y′=H, OR, N₃, NR′R″, SR′″,
   where R=H, benzyl, trimethylsilyl, tert-butyldimethylsilyl, tert-butyldiphenylsilyl, or acetate group,
   R′, R″ independently=H, alkyl, allyl,
   Bn, tosylate, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²=R³=H or CH₃,
   then R¹=

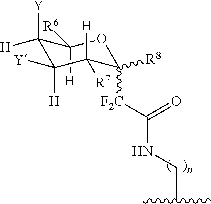

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula III:

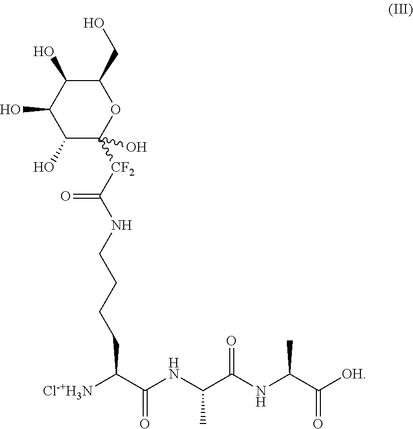

The use may be with from about 0.01 mg/ml to about 5 mg/ml of the compound of formula I, formula II or formula III.

The use may be with from about 1 mg/ml to about 5 mg/ml of the compound of formula I, formula II or formula III.

The use may be at least once, twice, three times, or four times per day.

According to another embodiment, there is provided a gem-difluorinated C-glycopeptide compound of general formula I, or a pharmaceutically acceptable base, addition salt with an acid, hydrate or solvate of the compound of general formula I for use in the treatment or prevention of dry eye disease in a subject in need thereof:

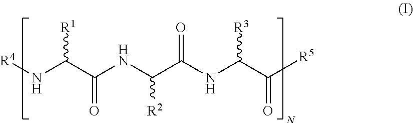

in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

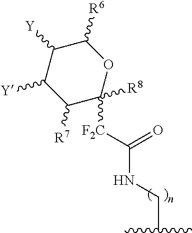

in which:
n is an integer between 3 and 4,
Y, Y′ are independent groups
   in which Y, Y′=H, OR, N₃, NR′R″, or SR′″,
      where R=H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″=H, alkyl, or acetate group,
R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   and
   if R¹=R²=H, CH₃, CH₂Ph, CH(CH₃)₂, CH₂CH(CH₃)₂, or CH(CH₃)CH₂CH₃ then R³=

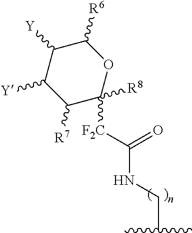

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

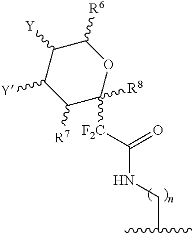

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²=R³=H, CH₃, CH(CH₃)₂, CH₂CH(CH₃)₂, or CH(CH₃)CH₂CH₃
   then R¹=

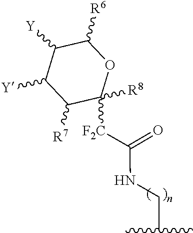

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject may be a human subject.

The compound of formula I may be a compound of formula II:

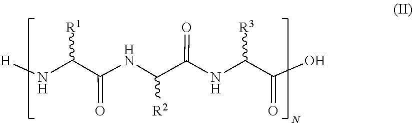

in which: N is an integer between 1 and 5,
   and
   R¹, R², R³ are independent groups in which two of R¹, R² and R³ are selected from H, CH₃ and the remaining R¹, R² and R³ is

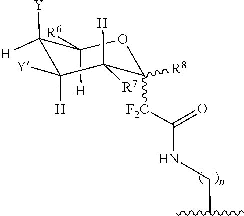

in which: n is an integer between 3 and 4,
   Y, Y′ are independent groups
      in which Y, Y′=H, OR, N₃, NR′R″ or SR′″,
         where R=H, benzyl, trimethylsilyl, tert-butyldimethylsilyl, tert-butyldiphenylsilyl, or acetate group,
         R′, R″ independently=H, alkyl, allyl, benzyl, tosylate group, C(═O)-alkyl, or C(═O)—Bn,
         R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R³ is a hydrogen atom H or a free or protected alcohol function,
      and
      if R¹=R²=H or CH₃,
      then R³=

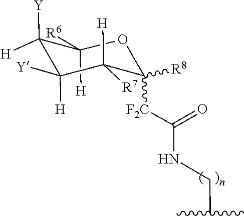

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹=R³=H or CH₃,
   then R²=

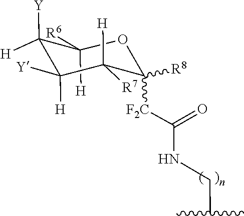

in which: n is an integer between 3 and 4,
Y, Y′ are independent groups
   in which Y, Y′=H, OR, N₃, NR′R″, SR′″,
   where R=H, benzyl, trimethylsilyl, tert-butyldimethylsilyl, tert-butyldiphenylsilyl, or acetate group,
   R′, R″ independently=H, alkyl, allyl,
   Bn, tosylate, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²=R³=H or CH₃,
   then R¹=

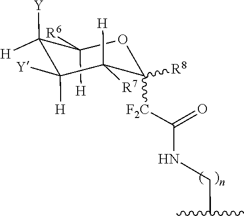

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula III:

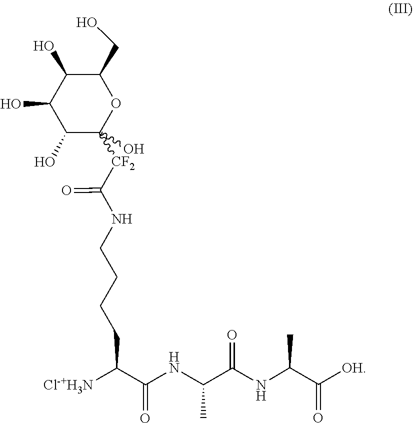

The use may be with from about 0.01 mg/ml to about 5 mg/ml of the compound of formula I, formula II or formula III.

The use may be with from about 1 mg/ml to about 5 mg/ml of the compound of formula I, formula II or formula III.

The use may be at least once, twice, three times, or four times per day.

According to another embodiment, there is provided the use of a gem-difluorinated C-glycopeptide compound of general formula I, or a pharmaceutically acceptable base, addition salt with an acid, hydrate or solvate of the compound of general formula I in the manufacture of a medicament for the treatment or prevention of dry eye disease in a subject in need thereof:

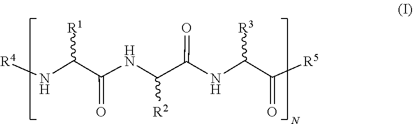

in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

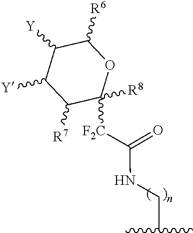

in which:
   n is an integer between 3 and 4,
   Y, Y′ are independent groups
      in which Y, Y′=H, OR, N₃, NR′R″, or SR′″,
         where R=H, benzyl, trimethylsilyl, tert-butyldimethylsilyl, tert-butyldiphenylsilyl or acetate group,
         R′, R″ independently=H, alkyl, allyl, benzyl, tosylate group, C(═O)-alkyl or C(═O)—Bn,
         R′″=H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃
      then R³=

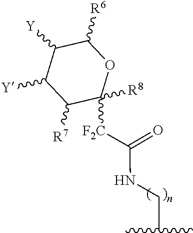

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group, R³ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

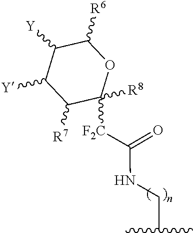

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²=R³=H, CH₃, CH(CH₃)₂, CH₂CH(CH₃)₂, or CH(CH₃)CH₂CH₃
   then R¹=

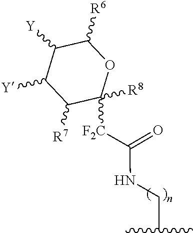

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject may be a human subject.

The compound of formula I may be a compound of formula II:

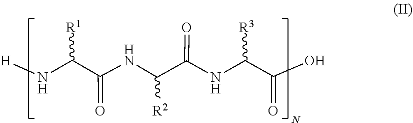

in which: N is an integer between 1 and 5,
   and
   R¹, R², R³ are independent groups in which two of R¹, R² and R³ are selected from H, CH₃ and the remaining R¹, R² and R³ is

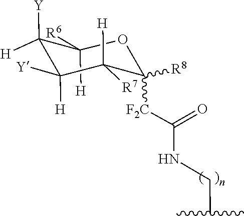

in which: n is an integer between 3 and 4,
   Y, Y′ are independent groups
      in which Y, Y′=H, OR, N₃, NR′R″ or SR′″,
         where R=H, benzyl, trimethylsilyl, tert-butyldimethylsilyl, tert-butyldiphenylsilyl, or acetate group,
         R′, R″ independently=H, alkyl, allyl, benzyl, tosylate group, C(═O)-alkyl, or C(═O)—Bn,
         R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R⁸ is a hydrogen atom H or a free or protected alcohol function,
      and
      if R¹=R²=H or CH₃,
      then R³=

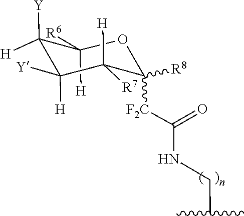

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹=R³=H or CH₃,
   then R²=

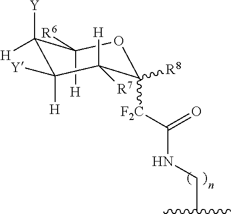

in which: n is an integer between 3 and 4,
Y, Y′ are independent groups
   in which Y, Y′=H, OR, N₃, NR′R″, SR′″,
   where R=H, benzyl, trimethylsilyl, tert-butyldimethylsilyl, tert-butyldiphenylsilyl, or acetate group,
   R′, R″ independently=H, alkyl, allyl,
   Bn, tosylate, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²=R³=H or CH₃,
   then R¹=

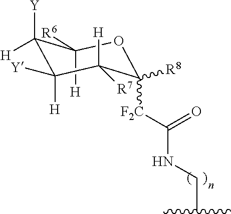

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula III:

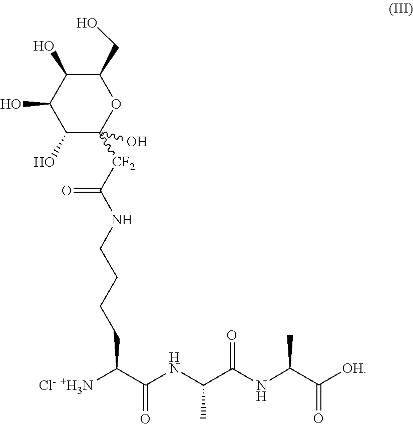

The use may be with from about 0.01 mg/ml to about 5 mg/ml of the compound of formula I, formula II or formula III.

The use may be with from about 1 mg/ml to about 5 mg/ml of the compound of formula I, formula II or formula III.

The medicament may be for use at least once, twice, three times, or four times per day.

According to another embodiment, there is provided a method for the treatment or prevention of a retinal degenerative disease, an ocular inflammation, or a combination thereof, in a subject in need thereof comprising the step of:
a) administering to an eye of the subject an effective amount of a gem-difluorinated C-glycopeptide compound of general formula I, or a pharmaceutically acceptable base, addition salt with an acid, hydrate or solvate of the compound of general formula I:

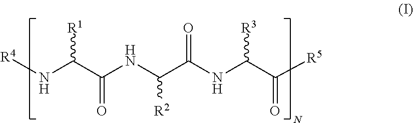

in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain
and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

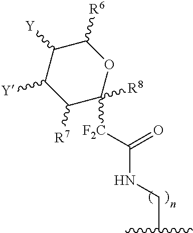

in which:
   n is an integer between 3 and 4,
   Y, Y′ are independent groups
      in which Y, Y′=H, OR, N₃, NR′R″, or SR′″,
         where R=H, benzyl, trimethylsilyl, tert-butyldimethylsilyl, tert-butyldiphenylsilyl or acetate group,
         R′, R″ independently=H, alkyl, allyl, benzyl, tosylate group, C(═O)-alkyl or C(═O)—Bn,
         R′″=H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃
      then R³=

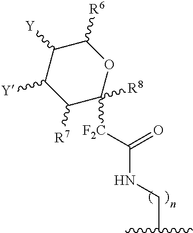

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn, R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

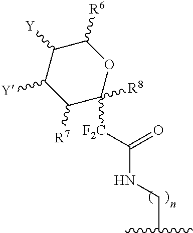

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn, R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²=R³=H, CH₃, CH(CH₃)₂, CH₂CH(CH₃)₂, or CH(CH₃)CH₂CH₃
   then R¹=

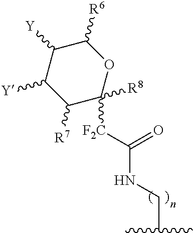

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject may be a human subject.

The compound of formula I may be a compound of formula II:

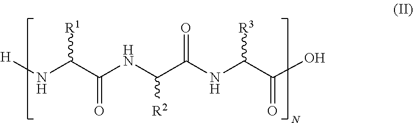

in which: N is an integer between 1 and 5,
   and
   R¹, R², R³ are independent groups in which two of R¹, R² and R³ are selected from H, CH₃ and the remaining R¹, R² and R³ is

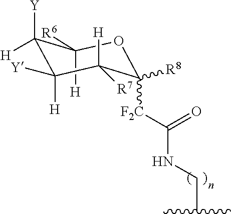

in which: n is an integer between 3 and 4,
   Y, Y′ are independent groups
      in which Y, Y′=H, OR, N₃, NR′R″ or SR′″,
         where R=H, benzyl, trimethylsilyl, tert-butyldimethylsilyl, tert-butyldiphenylsilyl, or acetate group,
         R′, R″ independently=H, alkyl, allyl, benzyl, tosylate group, C(═O)-alkyl, or C(═O)—Bn,
         R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R³ is a hydrogen atom H or a free or protected alcohol function,
      and
      if R¹=R²=H or CH₃,
      then R³=

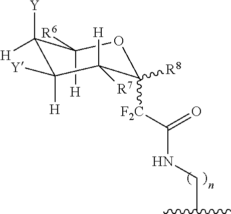

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹=R³=H or CH₃,
   then R²=

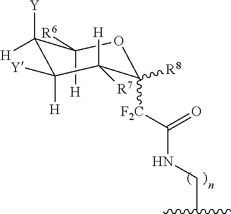

in which: n is an integer between 3 and 4,
Y, Y′ are independent groups
   in which Y, Y′=H, OR, N₃, NR′R″, SR′″,
   where R=H, benzyl, trimethylsilyl, tert-butyldimethylsilyl, tert-butyldiphenylsilyl, or acetate group,
   R′, R″ independently=H, alkyl, allyl,
   Bn, tosylate, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²=R³=H or CH₃,
   then R¹=

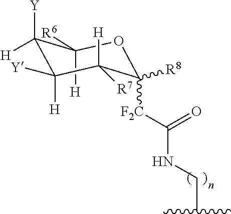

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula III:

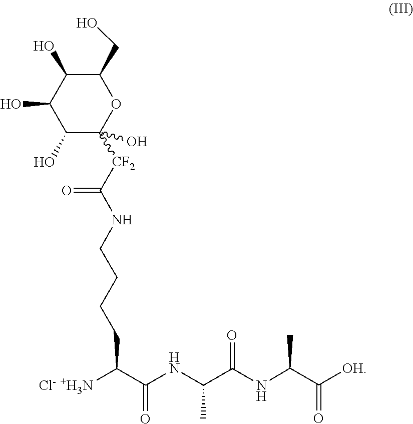

The effective amount may be from about from about 0.01 mg/ml to about 5 mg/ml of the compound of formula I, formula II or formula III.

The effective amount may be with from about 1 mg/ml to about 5 mg/ml of the compound of formula I, formula II or formula III.

The administration may be at least once, twice, three times, or four times per day.

The retinal degenerative disease may be age-related macular degeneration (AMD), diabetic retinopathy, retinitis pigmentosa (RP), retinal vein occlusion, retinal vasculitis, or sarcoidosis.

According to another embodiment, there is provided a use of a gem-difluorinated C-glycopeptide compound of general formula I, or a pharmaceutically acceptable base, addition salt with an acid, hydrate or solvate of the compound of general formula I for the treatment or prevention of a retinal degenerative disease, an ocular inflammation, or a combination thereof, in a subject in need thereof:

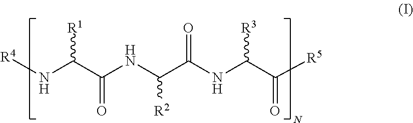

in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

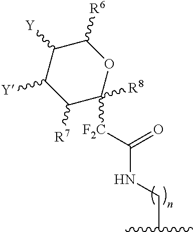

in which:
   n is an integer between 3 and 4,
   Y, Y′ are independent groups
      in which Y, Y′=H, OR, N₃, NR′R″, or SR′″,
         where R=H, benzyl, trimethylsilyl, tert-butyldimethylsilyl, tert-butyldiphenylsilyl or acetate group,
         R′, R″ independently=H, alkyl, allyl, benzyl, tosylate group, C(═O)-alkyl or C(═O)—Bn,
         R′″=H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃ then R³=

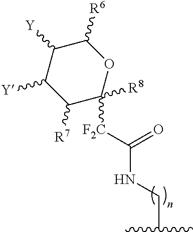

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃ then R²=

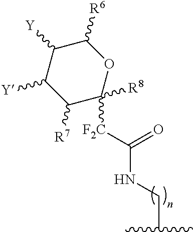

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²=R³=H, CH₃, CH(CH₃)₂, CH₂CH(CH₃)₂, or CH(CH₃)CH₂CH₃
   then R¹=

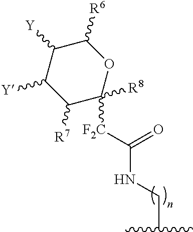

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject may be a human subject.

The compound of formula I may be a compound of formula II:

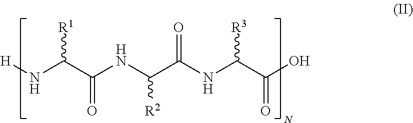

in which: N is an integer between 1 and 5,
   and
   R¹, R², R³ are independent groups in which two of R¹, R² and R³ are selected from H, CH₃ and the remaining R¹, R² and R³ is

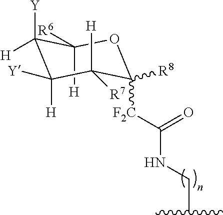

in which: n is an integer between 3 and 4,
   Y, Y′ are independent groups
      in which Y, Y′=H, OR, N₃, NR′R″ or SR′″,
      where R=H, benzyl, trimethylsilyl, tert-butyldimethylsilyl, tert-butyldiphenylsilyl, or acetate group,
      R′, R″ independently=H, alkyl, allyl, benzyl, tosylate group, C(═O)-alkyl, or C(═O)—Bn,
      R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R⁸ is a hydrogen atom H or a free or protected alcohol function,
      and
      if R¹=R²=H or CH₃,
      then R³=

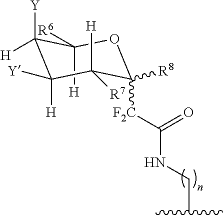

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹=R³=H or CH₃,
   then R²=

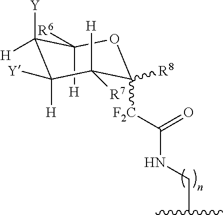

in which: n is an integer between 3 and 4,
Y, Y′ are independent groups
   in which Y, Y′=H, OR, N₃, NR′R″, SR′″,
   where R=H, benzyl, trimethylsilyl, tert-butyldimethylsilyl, tert-butyldiphenylsilyl, or acetate group,
   R′, R″ independently=H, alkyl, allyl,
   Bn, tosylate, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²=R³=H or CH₃,
   then R¹=

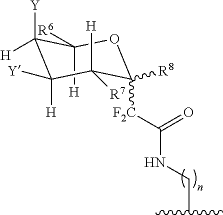

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula III:

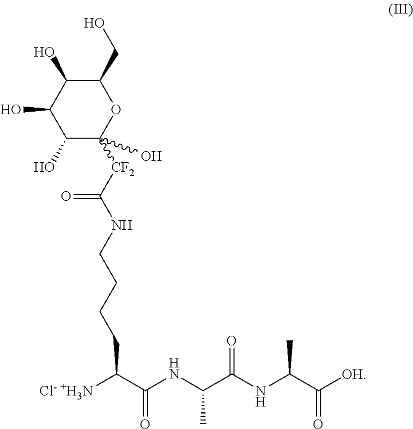

The use may be with from about 0.01 mg/ml to about 5 mg/ml of the compound of formula I, formula II or formula III.

The use may be with from about 1 mg/ml to about 5 mg/ml of the compound of formula I, formula II or formula III.

The use may be at least once, twice, three times, or four times per day.

The retinal degenerative disease is age-related macular degeneration (AMD), diabetic retinopathy, retinitis pigmentosa (RP), retinal vein occlusion, retinal vasculitis, or sarcoidosis.

The following terms are defined below.

The term «time sufficient» as used herein is intended to mean the time it takes for treatment or prevention of the dry eye disease symptoms. This time will be variable and depend upon the type of dry eye disease. It may range from a few minutes, a day or a few days, a week or a few weeks to months.

The terms “inhibit”, “inhibition” or “inhibiting”; “prevent” or “prevention” as used herein in the context of the invention means to slow, hinder, restrain reduce or prevent. For example, “prevention of dry eye disease” is used herein means to slow, hinder, restrain, reduce or prevent dry eye disease.

The term «contacting» as used herein is intended to mean touching the organs, portion(s) thereof, tissues and/or cells with the compound of the present invention, for a sufficient amount of time to provide the effect(s) imparted by the compounds. According to an embodiment, contacting is used herein to mean making contact with an eye.

The term «administering» as used herein is intended to mean dispense or apply (a remedy or drug; i.e. a compound of formula I, II or Ill). According to an embodiment, administering is used herein to mean administering to an eye.

The term a «subject» is preferably a human subject but can also be any mammal, including an animal model. Mammals of interest include, but are not limited to: rodents, e.g. mice, rats; livestock, e.g. pigs, horses, cows, etc., pets, e.g. dogs, cats; and primates. A subject may also be referred to herein as a “patient”.

The term «composition» as used herein is intended to encompass a product comprising the specified ingredients in the specified amounts, as well as any product which results, directly or indirectly, from combination of the specified ingredients in the specified amounts. Such term in relation to pharmaceutical composition or other compositions in general, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions or other compositions in general of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof. According to some embodiments, the composition may be an ophthalmic composition, which may be formulated as any one of a solution, an ointment, a cream, a lotion, eye drops or an eye gel.

The terms “dry eye” or “dry eye disease” as used herein is intended to encompass dry eye syndrome (DES), also known as keratoconjunctivitis sicca (KCS—“dryness of the cornea and conjunctiva” in Latin) which is the condition of having dry eyes. Other associated symptoms include irritation, redness, discharge, and easily fatigued eyes. Blurred vision may also occur. The symptoms can range from mild and occasional to severe and continuous. Scarring of the cornea may occur in some cases without treatment. Dry eye occurs when either the eye does not produce enough tears or when the tears evaporate too quickly. This can result from contact lens use, meibomian gland dysfunction, allergies, pregnancy, Sjögren syndrome, vitamin A deficiency, LASIK surgery, and certain medications such as antihistamines, some blood pressure medication, hormone replacement therapy, and antidepressants. Chronic conjunctivitis such as from tobacco smoke exposure or infection may also lead to the condition. Dry eye disease is a common eye disease. It affects 5-34% of people to some degree depending on the population looked at. Among older people it affects up to 70%.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂⁻), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus include heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

The expression “an ocular inflammation” is intended to mean inflammation of the eye, and can refer to inflammation of the eyelids or inflammation of the uvea (middle layer of the eye). Symptoms of inflammation to the eyelids include crusting, swelling, redness, and itching. Inflammation of the uvea includes blurred vision, eye pain, eye redness, and light sensitivity. Eyelid inflammation can be a symptom of a bacterial infection such as a stye. Uveitis is inflammation of the middle layer of the eyeball, which consists of the iris, ciliary body and choroid. Collectively, these structures are called the uvea. Uveitis can have many causes, including eye injury and inflammatory diseases. Exposure to toxic chemicals such as pesticides and acids used in manufacturing processes also can cause uveitis. The type of uveitis you have is classified by where inflammation occurs in the uvea: Anterior uveitis is inflammation of the iris (iritis) or the iris and ciliary body. Intermediate uveitis is inflammation of the ciliary body. Posterior uveitis is inflammation of the choroid. Diffuse uveitis (also called pan-uveitis) is inflammation of all areas of the uvea. Blepharitis is eyelid inflammation. There are two types of eyelid inflammation: Anterior eye inflammation occurs on the outside of your eye where your eyelashes are located. Dandruff on your eyebrows and allergic reactions in your eyes can cause anterior eyelid inflammation. Posterior eyelid inflammation occurs on the inner edge of eyelids closest to your eye. A malfunctioning oil gland behind your eyelash follicles usually causes this form of inflammation.

The expression “retinal degeneration” is a retinopathy which consists in the deterioration of the retina caused by the progressive death of its cells. There are several reasons for retinal degeneration, including artery or vein occlusion, diabetic retinopathy, retrolental fibroplasia, retinopathy of prematurity, or disease (usually hereditary).

The expression “retinal degenerative disease” is a disease caused by or from retinal degeneration.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1A illustrates Corneal Permeability after acute DED induction. Mean±SEM; Unpaired student's t-test; *, p≤0.05; **, p≤0.01; ***, p≤0.001.

FIG. 1B illustrates Corneal Permeability after acute DED induction. Mean±SEM; Unpaired student's t-test; *, p≤0.05; **, p≤0.01; ***, p≤0.001.

FIG. 2 illustrates conjunctival goblet cell density, as histogram (left) and dot and whisker plot (right).

FIG. 3A illustrates Corneal Permeability after acute DED induction. Mean±SEM; Unpaired student's t-test; *, p≤0.05; **, p≤0.01; ***, p≤0.001.

FIG. 3B illustrates Corneal Permeability after acute DED induction. Mean±SEM; Unpaired student's t-test; *, p≤0.05; **, p≤0.01; ***, p≤0.001.

FIG. 4A illustrates the CD4+ T cells in the conjunctival epithelium after acute DED induction.

FIG. 4B illustrates the CD4+ T cells in the conjunctival epithelium after acute DED induction.

It will be noted that throughout the appended drawings, like features are identified by like reference numerals.

### DETAILED DESCRIPTION

In embodiments there is disclosed a for the treatment or prevention of dry eye disease in a subject in need thereof comprising the step of:

a) administering to an eye of the subject an effective amount of a gem-difluorinated C-glycopeptide compound of general formula I, or a pharmaceutically acceptable base, addition salt with an acid, hydrate or solvate of the compound of general formula I

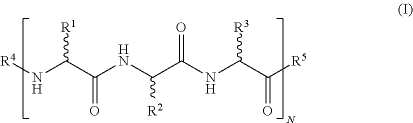

in which:
N is an integer between 1 and 5,
   R⁴=H, AA₁, or AA₁-AA₂,
   R⁵=OH, AA₁, or AA₁-AA₂,
   AA₁ and AA₂ independently represent amino acids with a non-polar side chain
   and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

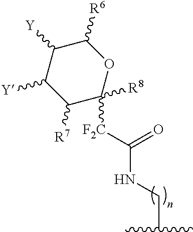

in which:
n is an integer between 3 and 4,
   Y, Y′ are independent groups
      in which Y, Y′=H, OR, N₃, NR′R″, or SR′″,
      where R=H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″=H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃
      then R³=

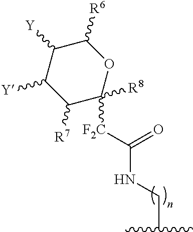

in which: n is an integer between 3 and 4,
Y, Y′ are independent groups
in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
   R⁶ is H, CH₃, CH₂OH, CH₂-glycoside group, or CH₂-OGP in which GP is a protector group selected
   from alkyl, benzyl, trimethylsilyl, tertbutyldimethylsilyl, tert-butyldiphenylsilyl, or acetate group,
   R⁷=OH, OGP′, NH₂, N₃, NHGP′, or NGP′GP″ in which GP′ and GP″ are independently selected from
   alkyl, benzyl, trimethylsilyl, tertbutyldimethylsilyl, tert-butyldiphenylsilyl, or acetate group,
   R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

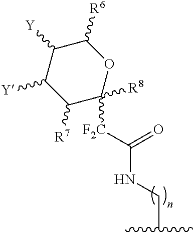

in which: n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂-OGP in which GP is a protector group selected
from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from
alkyl, benzyl, trimethylsilyl, tertbutyldimethylsilyl, tert-butyldiphenylsilyl, or acetate group,
R⁸ is a hydrogen atom H or a free or protected alcohol function,
if R²=R³=H, CH₃, CH(CH₃)₂, CH₂CH(CH₃)₂, or CH(CH₃)CH₂CH₃
then R¹=

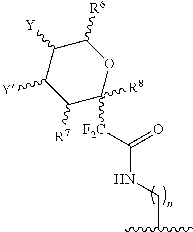

in which: n is an integer between 3 and 4,
Y, Y′ are independent groups
in which Y, Y′ H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl,
tert-butyldiphenylsilyl, or acetate group,
R⁸ is a hydrogen atom H or a free or protected alcohol function

The subject may be a human subject.

The compound of formula I may be a compound of formula II:

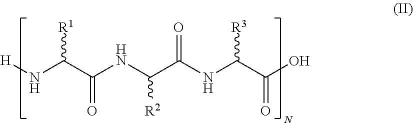

in which: N is an integer between 1 and 5,
and
R¹, R², R³ are independent groups in which two of R¹, R² and R³ are selected from H, CH₃ and the remaining R¹, R² and R³ is

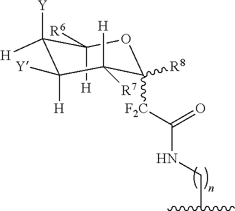

in which: n is an integer between 3 and 4,
Y, Y′ are independent groups
   in which Y, Y′=H, OR, N₃, NR′R″ or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R⁸ is a hydrogen atom H or a free or protected alcohol function,
   and
   if R¹=R²=H or CH₃,
   then R³=

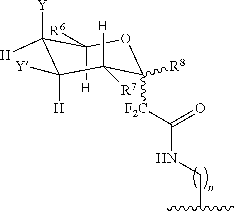

in which: n is an integer between 3 and 4,
Y, Y′ are independent groups
in which Y, Y′=H, OR, N₃, NR′R″, or SR′″, where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R¹=R³=H or CH₃,
then R²=

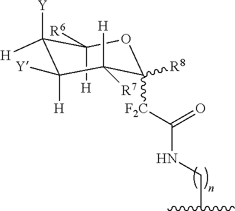

in which: n is an integer between 3 and 4,
Y, Y′ are independent groups
in which Y, Y′=H, OR, N₃, NR′R″, SR′″,
where R=H, benzyl, trimethylsilyl, tert-butyldimethylsilyl, tert-butyldiphenylsilyl, or acetate group,
R′, R″ independently=H, alkyl, allyl,
Bn, tosylate, C(═O)-alkyl, or C(═O)—Bn,
R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R²=R³=H or CH₃,
then R¹=

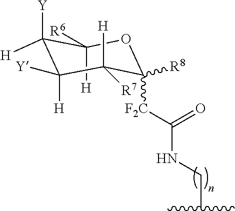

in which: n is an integer between 3 and 4,
Y, Y′ are independent groups
in which Y, Y′ H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom or a free or protected alcohol function.

The compound of formula I may be a compound of formula III:

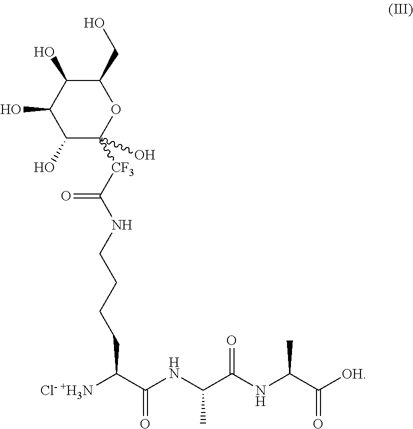

The effective amount may be from about 0.01 mg/ml to about 5 mg/ml; or from about 0.1 mg/ml to about 5 mg/ml; or from about 0.5 mg/ml to about 5 mg/ml; or from about 1 mg/ml to about 5 mg/ml; or from about 3 mg/ml to about 5 mg/ml; or from about 0.01 mg/ml to about 3 mg/ml, or from about 0.1 mg/ml to about 3 mg/ml, or from about 0.5 mg/ml to about 3 mg/ml, or from about 1 mg/ml to about 3 mg/ml, or from about 0.01 mg/ml to about 1 mg/ml; or from about 0.1 mg/ml to about 1 mg/ml; or from about 0.5 mg/ml to about 1 mg/ml; or from about 0.01 mg/ml to about 0.5 mg/ml; or from about 0.1 mg/ml to about 0.5 mg/ml; or from about 0.01 mg/ml to about 0.1 mg/ml; or about 3 mg/ml of the compound of formula i, formula II or formula III. According to embodiments, the amounts above are considered to be therapeutically effective amounts for the purpose of the present inventions.

The administration may be at least once, twice, three times, four times, or more times per day.

According to another embodiment, there is provided a use of a gem-difluorinated C-glycopeptide compound of general formula i, or a pharmaceutically acceptable base, addition salt with an acid, hydrate or solvate of the compound of general formula I for the treatment or prevention of dry eye disease in a subject in need thereof:

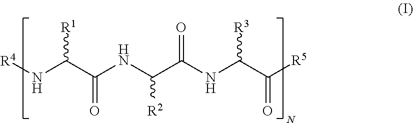

in which:
N is an integer between 1 and 5,
   R⁴=H, AA₁, or AA₁-AA₂,
   R⁵=OH, AA₁, or AA₁-AA₂,
   AA₁ and AA₂ independently represent amino acids with a non-polar side chain
   and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

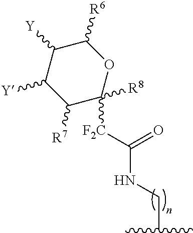

in which:
n is an integer between 3 and 4,
   Y, Y′ are independent groups
      in which Y, Y′=H, OR, N₃, NR′R″, or SR′″,
      where R=H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″=H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃
      then R³=

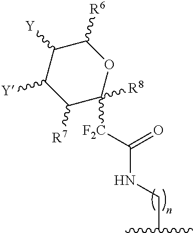

in which: n is an integer between 3 and 4,
Y, Y′ are independent groups
in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
   R⁶ is H, CH₃, CH₂OH, CH₂-glycoside group, or CH₂—OGP in which GP is a protector group selected from alkyl, benzyl, trimethylsilyl, tertbutyldimethylsilyl, tert-butyldiphenylsilyl, or acetate group,
   R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
   R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

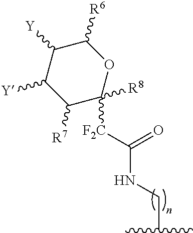

in which: n is an integer between 3 and 4,
Y, Y′ are independent groups
in which Y, Y′=H, OR, N₃, NR′R″, or SR′″, where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group, R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²=R³=H, CH₃, CH(CH₃)₂, CH₂CH(CH₃)₂, or CH(CH₃)CH₂CH₃
then R¹=

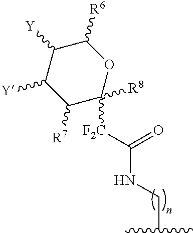

in which: n is an integer between 3 and 4,
Y, Y′ are independent groups
in which Y, Y′ H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

According to another embodiment, there is provided a gem-difluorinated C-glycopeptide compound of general formula I, or a pharmaceutically acceptable base, addition salt with an acid, hydrate or solvate of the compound of general formula I for use in the treatment or prevention of dry eye disease in a subject in need thereof:

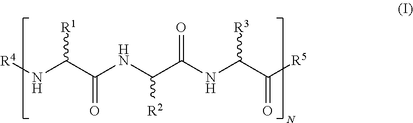

in which:
N is an integer between 1 and 5,
   R⁴=H, AA₁, or AA₁-AA₂,
   R⁵=OH, AA₁, or AA₁-AA₂,
   AA₁ and AA₂ independently represent amino acids with a non-polar side chain
   and
   R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

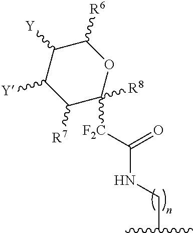

in which:
n is an integer between 3 and 4,
   Y, Y′ are independent groups
      in which Y, Y′=H, OR, N₃, NR′R″, or SR′″,
      where R=H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″=H, alkyl, or acetate group,
      R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
      R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
      R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃ then R³=

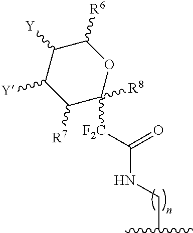

in which: n is an integer between 3 and 4,
Y, Y′ are independent groups
in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
   R⁶ is H, CH₃, CH₂OH, CH₂-glycoside group, or CH₂—OGP in which GP is a protector group selected from alkyl, benzyl, trimethylsilyl, tertbutyldimethylsilyl, tert-butyldiphenylsilyl, or acetate group,
   R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
   R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

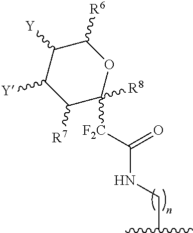

in which: n is an integer between 3 and 4,
Y, Y′ are independent groups
in which Y, Y′=H, OR, N₃, NR′R″, or SR′″, where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²=R³=H, CH₃, CH(CH₃)₂, CH₂CH(CH₃)₂, or CH(CH₃)CH₂CH₃
then R¹=

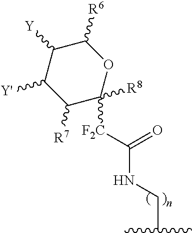

in which: n is an integer between 3 and 4,
Y, Y′ are independent groups
in which Y, Y′ H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

According to another embodiment, there is provided the use of a gem-difluorinated C-glycopeptide compound of general formula I, or a pharmaceutically acceptable base, addition salt with an acid, hydrate or solvate of the compound of general formula I in the manufacture of a medicament for the treatment or prevention of dry eye disease in a subject in need thereof:

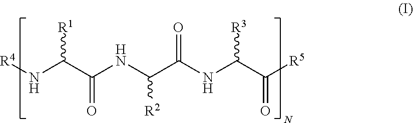

in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain
and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

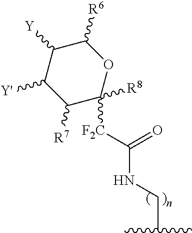

in which:
   n is an integer between 3 and 4,
   Y, Y′ are independent groups
      in which Y, Y′=H, OR, N₃, NR′R″, or SR′″,
      where R=H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″=H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃ then R³=

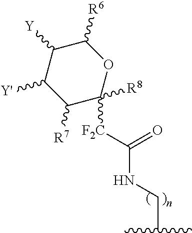

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

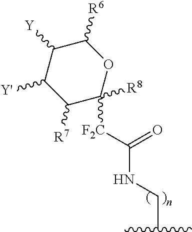

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²=R³=H, CH₃, CH(CH₃)₂, CH₂CH(CH₃)₂, or CH(CH₃)CH₂CH₃
   then R¹=

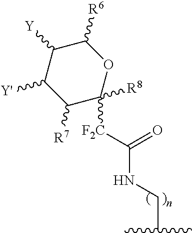

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

The subject may be a human subject.

The compound of formula I may be a compound of formula II:

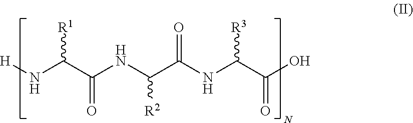

in which: N is an integer between 1 and 5,
   and
   R¹, R², R³ are independent groups in which two of R¹, R² and R³ are selected from H, CH₃ and the remaining R¹, R² and R³ is

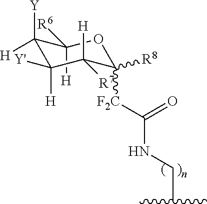

in which: n is an integer between 3 and 4,
   Y, Y′ are independent groups
      in which Y, Y′=H, OR, N₃, NR′R″ or SR′″,
         where R=H, benzyl, trimethylsilyl, tert-butyldimethylsilyl, tert-butyldiphenylsilyl, or acetate group,
         R′, R″ independently=H, alkyl, allyl, benzyl, tosylate group, C(═O)-alkyl, or C(═O)—Bn,
         R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R⁸ is a hydrogen atom H or a free or protected alcohol function,
      and
      if R¹=R²=H or CH₃,
      then R³=

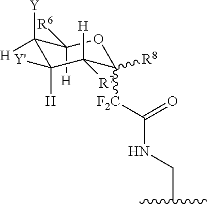

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹=R³=H or CH₃,
   then R²=

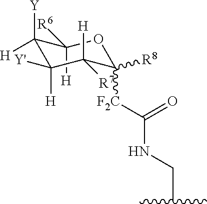

in which: n is an integer between 3 and 4,
Y, Y′ are independent groups
   in which Y, Y′=H, OR, N₃, NR′R″, SR′″,
   where R=H, benzyl, trimethylsilyl, tert-butyldimethylsilyl, tert-butyldiphenylsilyl, or acetate group,
   R′, R″ independently=H, alkyl, allyl,
   Bn, tosylate, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²=R³=H or CH₃,
   then R¹=

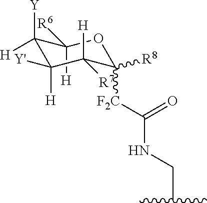

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula III:

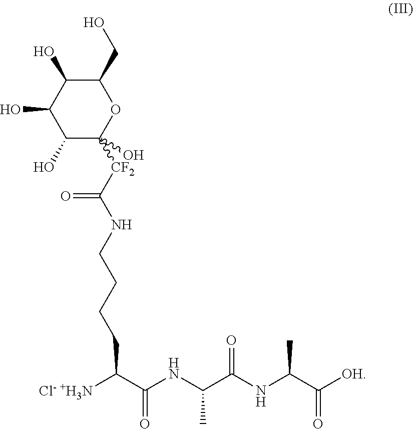

The use may be with from about 0.01 mg/ml to about 5 mg/ml; or from about 0.1 mg/ml to about 5 mg/ml; or from about 0.5 mg/ml to about 5 mg/ml; or from about 1 mg/ml to about 5 mg/ml; or from about 3 mg/ml to about 5 mg/ml; or from about 0.01 mg/ml to about 3 mg/ml, or from about 0.1 mg/ml to about 3 mg/ml, or from about 0.5 mg/ml to about 3 mg/ml, or from about 1 mg/ml to about 3 mg/ml, or from about 0.01 mg/ml to about 1 mg/ml; or from about 0.1 mg/ml to about 1 mg/ml; or from about 0.5 mg/ml to about 1 mg/ml; or from about 0.01 mg/ml to about 0.5 mg/ml; or from about 0.1 mg/ml to about 0.5 mg/ml; or from about 0.01 mg/ml to about 0.1 mg/ml; or about 3 mg/ml of the compound of formula I, formula II or formula III. According to embodiments, the amounts above are considered to be therapeutically effective amounts for the purpose of the present inventions.

The medicament may be for use at least once, twice, three times, four times or more times per day.

According to another embodiment, there is disclosed a method for the treatment or prevention of a retinal degenerative disease, an ocular inflammation, or a combination thereof, in a subject in need thereof comprising the step of:
a) administering to an eye of the subject an effective amount of a gem-difluorinated C-glycopeptide compound of general formula I, or a pharmaceutically acceptable base, addition salt with an acid, hydrate or solvate of the compound of general formula I:

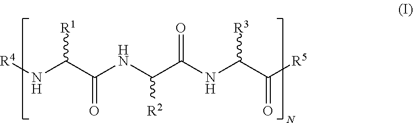

in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is

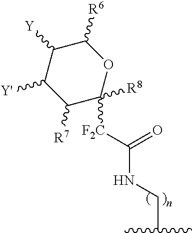

in which:
n is an integer between 3 and 4,
Y, Y′ are independent groups
   in which Y, Y′=H, OR, N₃, NR′R″, or SR′″,
      where R=H, benzyl, trimethylsilyl, tert-butyldimethylsilyl, tert-butyldiphenylsilyl or acetate group,
      R′, R″ independently=H, alkyl, allyl, benzyl, tosylate group, C(═O)-alkyl or C(═O)—Bn,
      R′″=H, alkyl, or acetate group,
   R⁶ is H, CH₃, CH₂OH, CH₂-glycoside group or CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom H or a free or protected alcohol function,
      and
      if R¹=R²=H, CH₃, CH₂Ph, CH(CH₃)₂, CH₂CH(CH₃)₂, or CH(CH₃)CH₂CH₃ then R³=

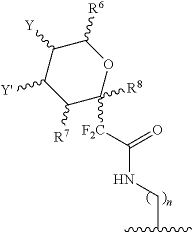

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R¹=R³=H, CH₃, CH₂Ph, CH(CH₃)₂, CH₂CH(CH₃)₂, or CH(CH₃)CH₂CH₃
   then R²=

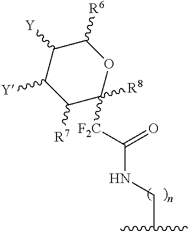

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
   if R²=R³=H, CH₃, CH(CH₃)₂, CH₂CH(CH₃)₂, or CH(CH₃)CH₂CH₃
   then R¹=

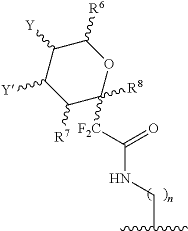

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is H, CH₃, CH₂OH, CH₂-glycoside group, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

According to another embodiment, there is disclosed the use of a gem-difluorinated C-glycopeptide compound of general formula I, or a pharmaceutically acceptable base, addition salt with an acid, hydrate or solvate of the compound of general formula I for the treatment or prevention of a retinal degenerative disease, an ocular inflammation, or a combination thereof, in a subject in need thereof.

The subject may be a human subject.

The compound of formula I may be a compound of formula II:

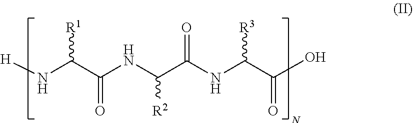

in which: N is an integer between 1 and 5,
   and
   R¹, R², R³ are independent groups in which two of R¹, R² and R³ are selected from H, CH₃ and the remaining R¹, R² and R³ is

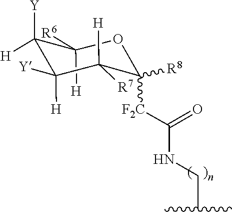

in which: n is an integer between 3 and 4,
   Y, Y′ are independent groups
      in which Y, Y′=H, OR, N₃, NR′R″ or SR′″,
         where R=H, benzyl, trimethylsilyl, tert-butyldimethylsilyl, tert-butyldiphenylsilyl, or acetate group,
         R′, R″ independently=H, alkyl, allyl, benzyl, tosylate group, C(═O)-alkyl, or C(═O)—Bn,
         R′″=H, alkyl, or acetate group,
   R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
   R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
   R⁸ is a hydrogen atom H or a free or protected alcohol function,
      and
      if R¹=R²=H or CH₃,
      then R³=

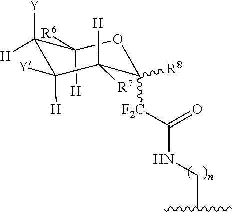

in which: n is an integer between 3 and 4,
Y, Y′ are independent groups
   in which Y, Y′=H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R¹=R³=H or CH₃,
   then R²=

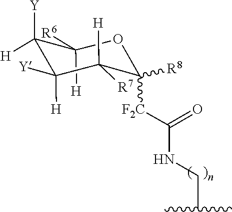

in which: n is an integer between 3 and 4,
Y, Y′ are independent groups
   in which Y, Y′=H, OR, N₃, NR′R″, SR′″,
   where R=H, benzyl, trimethylsilyl, tert-butyldimethylsilyl, tert-butyldiphenylsilyl, or acetate group,
   R′, R″ independently=H, alkyl, allyl,
   Bn, tosylate, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
   if R²=R³=H or CH₃,
   then R¹=

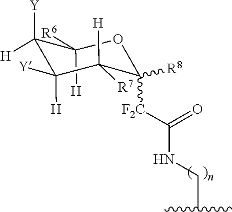

in which: n is an integer between 3 and 4,
Y, Y′ are independent groups
   in which Y, Y′ H, OR, N₃, NR′R″, or SR′″,
   where R=H, benzyl, trimethylsilyl, tert-butyldimethylsilyl, tert-butyldiphenylsilyl, or acetate group,
   R′, R″ independently=H, alkyl, allyl, benzyl, tosylate group, C(═O)-alkyl, or C(═O)—Bn,
   R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
   R⁸ is a hydrogen atom or a free or protected alcohol function.

The compound of formula I may be a compound of formula III:

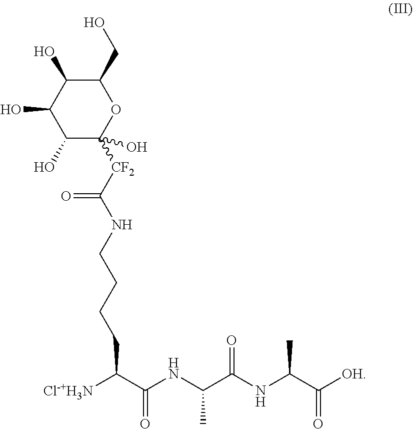

The effective amount may be from about 0.01 mg/ml to about 5 mg/ml; or from about 0.1 mg/ml to about 5 mg/ml; or from about 0.5 mg/ml to about 5 mg/ml; or from about 1 mg/ml to about 5 mg/ml; or from about 3 mg/ml to about 5 mg/ml; or from about 0.01 mg/ml to about 3 mg/ml, or from about 0.1 mg/ml to about 3 mg/ml, or from about 0.5 mg/ml to about 3 mg/ml, or from about 1 mg/ml to about 3 mg/ml, or from about 0.01 mg/ml to about 1 mg/ml; or from about 0.1 mg/ml to about 1 mg/ml; or from about 0.5 mg/ml to about 1 mg/ml; or from about 0.01 mg/ml to about 0.5 mg/ml; or from about 0.1 mg/ml to about 0.5 mg/ml; or from about 0.01 mg/ml to about 0.1 mg/ml; or about 3 mg/ml of the compound of formula I, formula II or formula III. According to embodiments, the amounts above are considered to be therapeutically effective amounts for the purpose of the present inventions.

The administration may be at least once, twice, three times, four times, or more times per day.

The retinal degenerative disease may be age-related macular degeneration (AMD), diabetic retinopathy, retinitis pigmentosa (RP), retinal vein occlusion, retinal vasculitis, or sarcoidosis.

The invention includes the compounds as shown, and also includes (where possible) individual diastereomers, enantiomers, and epimers of the compounds, and mixtures of diastereomers and/or enantiomers thereof including racemic mixtures. Although the specific stereochemistries disclosed herein are preferred, other stereoisomers, including diastereomers, enantiomers, epimers, and mixtures of these may also be useful. Inactive or less active diastereoisomers and enantiomers are useful for scientific studies relating to the targets and/or the mechanism of activation.

The compounds disclosed herein may be used in pharmaceutical compositions comprising (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound of Formula I, II or Ill, or a pharmaceutically acceptable salt thereof is the only active ingredient.

Compounds of structural Formula I, structural Formula II and/or structural Formula III may contain one or more asymmetric centers and can thus occur as racemates and racemic mixtures, single enantiomers, diastereomeric mixtures and individual diastereomers. The present invention is meant to comprehend all such isomeric forms of the compounds of structural Formula I, structural Formula II and/or structural Formula III.

Compounds of structural Formula I, structural Formula II and/or structural Formula III may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural Formula I, structural Formula II and/or structural Formula III may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of generic Formula I, Formula II and/or Formula III, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I, Formula II and/or Formula III. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I, Formula II and/or Formula III can be prepared without undue experimentation by conventional techniques well known to those skilled in the art.

### Salts and Formulations

It will be understood that, as used herein, references to the compounds of structural Formula I, Formula II and/or Formula III are meant to also include the pharmaceutically acceptable salts, and also salts that are not pharmaceutically acceptable when they are used as precursors to the free compounds or their pharmaceutically acceptable salts or in other synthetic manipulations. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural Formula I, Formula II and/or Formula III are included in the present invention as well.

According to an embodiment, the compounds of structural Formula I, Formula II and/or Formula III may be included in various formulations for use as medicaments.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the partial esters with ethylene oxide, for example polyoxyethylene sorbitan monooleate. The emulsions may also contain sweetening and flavouring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical compositions may be an ophthalmic composition, for example solutions, ointments, creams, lotions, eye ointments and, most preferably, eye drops or eye gels and can contain the appropriate conventional additives, including, for example, preservatives, solvents to assist drug penetration, and emollients in ointments and creams. When the pharmaceutical composition of the present invention for treating dry eye is used as an ophthalmic solution, it is provided in any dosage form which is used for ophthalmic solution, for example, an aqueous eye drop such as aqueous ophthalmic solution, aqueous suspended ophthalmic solution, viscous ophthalmic solution and solubilized ophthalmic solution, or a non-aqueous ophthalmic solution such as non-aqueous ophthalmic solution and non-aqueous suspended ophthalmic solution. Among these, the aqueous ophthalmic solution is preferable. When the pharmaceutical composition of the present invention for treating dry eye is prepared into an aqueous ophthalmic solution, various additives normally used in the aqueous ophthalmic solution are conveniently contained therein as long as the object of the present invention is not adversely affected. Examples of such the additives include buffers, isotonizing agents, preservatives, solubilizers (stabilizers), pH adjusting agents, thickeners and chelating agents. The buffers may be selected from but not limited by the group comprising a phosphate buffer, a borate buffer, a citrate buffer, a tartrate buffer, an acetate buffer (for example, sodium acetate) and an amino acid.

The isotonizing agents may be selected from but not limited by the group comprising sugars such as sorbitol, glucose and mannitol, polyhydric alcohols such as glycerin, polyethylene glycol and polypropylene glycol, and salts such as sodium chloride.

The preservatives may be selected from but not limited by the group comprising benzalkonium chloride, benzethonium chloride, alkyl paraoxybenzoates such as methyl paraoxybenzoate and ethyl paraoxybenzoate, benzyl alcohol, phenethyl alcohol, sorbic acid and salts thereof, thimerosal and chlorobutanol.

The solubilizers (stabilizers) may be selected from but not limited by the group comprising cyclodextrin and derivatives thereof, water-soluble polymers such as poly(vinylpyrrolidone), and surfactants such as polysorbate 80 (trade name: Tween 80).

The pH adjusting agents may be selected from but not limited by the group comprising hydrochloric acid, acetic acid, phosphoric acid, sodium hydroxide, potassium hydroxide and ammonium hydroxide.

The thickeners may be selected from but not limited by the group comprising hydroxyethylcellulose, hydroxypropylcellulose, methylcellulose, hydroxypropylmethylcellulose and carboxymethylcellulose and salts thereof.

The chelating agents may be selected from but not limited by the group comprising sodium edetate, sodium citrate and sodium condensed phosphate.

When the pharmaceutical composition of the present invention for treating dry eye is prepared into an ophthalmic ointment, a base compound must be present. The base of the ophthalmic ointment may be selected from but not limited by the group comprising purified lanolin, VASELINE®, plastibase, liquid paraffin and polyethylene glycol.

According to an embodiment, the eye(s) may be treated with a gem-difluorinated C-glycopeptide compound of general formula I—preferably, the compound of Formula II, and most preferably the compound of formula III in concentrations varying from about 0.01 mg/ml to about 5 mg/ml; or from about 0.1 mg/ml to about 5 mg/ml; or from about 0.5 mg/ml to about 5 mg/ml; or from about 1 mg/ml to about 5 mg/ml; or from about 3 mg/ml to about 5 mg/ml; or from about 0.01 mg/ml to about 3 mg/ml, or from about 0.1 mg/ml to about 3 mg/ml, or from about 0.5 mg/ml to about 3 mg/ml, or from about 1 mg/ml to about 3 mg/ml, or from about 0.01 mg/ml to about 1 mg/ml; or from about 0.1 mg/ml to about 1 mg/ml; or from about 0.5 mg/ml to about 1 mg/ml; or from about 0.01 mg/ml to about 0.5 mg/ml; or from about 0.1 mg/ml to about 0.5 mg/ml; or from about 0.01 mg/ml to about 0.1 mg/ml; or about 3 mg/ml. According to embodiments, the amounts above are considered to be therapeutically effective amounts for the purpose of the present inventions.

According to another embodiment, the eye(s) are contacted with the gem-difluorinated C-glycopeptide compound for a time sufficient to effect improvements on cell viability and survival rate. According to embodiments, the time sufficient may be from about 12 hours to 120 hours, or from about 12 hours to about 96 hours, or from about 12 hours to about 72 hours, or from about 12 hours to about 48 hours, or from about 12 hours to about 24 hours, or about 120 hours, or about 96 hours, or about 72 hours, or about 48 hours, or about 24 hours, or about 12 hours, or about 10 hours, or about 8 hours, or about 6 hours, or about 4 hours, or about 2 hours, or about 1 hour. In embodiments, the wherein the isolated neurosensory precursor cell is contacted with the compound for 1 hour, 55 mins, 50 mins, 45 mins, 40 mins, 35 mins, 30 mins, 25 mins, 20 mins, 15 mins, 10 mins, 5 mins, 4 mins, 3 mins, 2 mins, 1 mins, 45 secs, or 30 secs, or at least 1 hour, or at least 55 mins, or at least 50 mins, or at least 45 mins, or at least 40 mins, or at least 35 mins, or at least 30 mins, or at least 25 mins, or at least 20 mins, or at least 15 mins, or at least 10 mins, or at least 5 mins, or at least 4 mins, or at least 3 mins, or at least 2 mins, or at least 1 mins, or at least 45 secs, or at least 30 secs.

The present invention will be more readily understood by referring to the following examples which are given to illustrate the invention rather than to limit its scope.

### Example 1

### An Efficacy Evaluation of AAGP™ to Reduce Corneal Permeability in an Acute Mouse Model of Dry Eye Disease

The objective of this study was to determine the effect of 5% AAGP™, delivered four times a day via bilateral topical administration for seven consecutive days, on reduction of corneal permeability in an acute mouse model of dry eye disease induced by desiccating stress and scopolamine.

### LIST OF ABBREVIATIONS

BID Two times a day
BSS Balanced salt solution
DED Dry Eye Disease
DS Desiccating stress
OCT Optimal cutting temperature
OGD Oregon Green Dextran
NS Non-stressed
QID Four times a day
RH Relative humidity
RT Room temperature
SOP Standard Operating Procedure

Age and weight matched female C57BL/6J mice were divided into separate arms as shown in Table 1. On Days 1 through 7, the mice in study arms 4 and 5 received bilateral topical administration four times a day of vehicle (BSS; Arm 4), or 5% AAGP™ (Arm 5). On Days 4 through 7, the mice in study arm 3 received bilateral topical administration twice a day of 0.1% CsA-MiDROPS™ (cyclosporine A Microemulsion Drug Ocular Penetration System). The mice in study arms 1 and 2 were untreated throughout the study. On Days 4-8, the mice in study arms 2 through 5 were also kept in adverse environmental chambers (low humidity and constant airflow from a fan), and given subcutaneous injections of scopolamine hydrobromide occurred 4 times a day at 8:30 AM, 11:00 AM, 2:00 PM, and 4:30 PM, on Days 4 through 7, and once on Day 8 at 8:30 AM, to expose the animals to DS and induce acute DED. The mice in arm 1 were left untreated and unexposed to desiccating stress (DS) and untreated throughout the study.

Corneal permeability was assessed by Oregon Green Dextran (OGD) staining at baseline just prior to enrollment into study arms to determine if any mice met exclusion criteria. Corneal permeability was again measured on Day 8 for efficacy comparisons between mice in all study arms. At the completion of the in-life portion of the study, eye tissue was collected and processed for possible future histological analysis.

TABLE 1
Study design

| Arm | Induction | Treatment | Treatment Details | Assessment | Animal ID |
|---|---|---|---|---|---|
| 1 | None | None | n/a | Quantification of | 21230, 21231, 21233, |
| | | n = 5 | | corneal permeability | 21234, 21235 |
| 2 | Dry Eye | None | n/a | in all eyes at Day 8; | 21236, 21237, 21238, |
| | Disease | n = 5 | | Enucleation and | 21240 |
| 3 | Dry Eye | 0.1% CsA- | Bilateral topical | collection of eyes for | 21241, 21242, 21243, |
| | Disease | MIDROPS ™ | instillation, BID, on | histological | 21244, 21245, 21246, |
| | | n = 10 | Days 4-7 | examination on Day 8 | 21247, 21248, 21249, |
| | | | (2 μl/instillation) | | 21250 |
| 4 | Dry Eye | Vehicle (BSS) | Bilateral topical | | 21251, 21252, 21253, |
| | Disease | n = 10 | instillation, QID, on | | 21254, 21255, 21256, |
| | | | Days 1-7 | | 21257, 21259, 21260, |
| | | | (3 μl/instillation) | | 21261 |
| 5 | Dry Eye | 5% AAGP ™ | | | 21262, 21263, 21264, |
| | Disease | n = 10 | | | 21265, 21267, 21268, |
| | | | | | 21269, 21270, 21271, |
| | | | | | 21272 |

Study Objective

Efficacy Assessment

To determine the effect of 5% AAGP™ to reduce corneal permeability after repeat topical instillation in an acute mouse model of dry eye disease induced by desiccating stress and scopolamine.

Test Materials and Equipment

Test Article(s) Preparation and Storage

AAGP™ was provided as a powder of ˜500 mg and stored at −20° C. upon arrival. The test article was kept protected from light throughout the study. AAGP™ was formulated in BSS and aliquoted into daily use aliquots as follows:
1) AAGP™ powder was removed from −20° C., placed in a desiccating chamber, and brought to room temperature for ˜30 minutes.
2) The required amount of AAGP™ was weighed and into an appropriate container using a potency value of 87.79% (AAGP™ Lot number PD150709-1R).
3) 80% of the final required volume of vehicle (BSS sterile irrigating solution, Alcon, cat #0065079550) was added.
4) The solution was stirred until complete dissolution of AAGP™ 5) Measure pH and adjust to 5-8 by adding by slowly pipetting 1 N NaOH solution. Record the initial and adjusted pH values.
6) Transfer the AAGP™ formulation into a volumetric flask and add vehicle to the required final volume, to achieve the correct final concentration.
7) Measure and record the osmolarity and viscosity of the final solution.
8) Sterilize the solution by filtration through a 0.22 μm GV (PVDF) filter (Millipore) into a sterile vial.
9) Aliquot into daily use vials for administration.
10) Store the formulations at 4° C.±2° C. until the day of administration.

Test System

Species, Strain, Sex, Age, Weight, Supplier
Species: Mouse
Strain: C57BL/6J
Sex: Female
Age Range: 6 weeks old at time of arrival to test facility
Weight: >15-25 g
Supplier: Jackson Laboratories
Number of Study Animals: 40
Number of Spare Animals 0

Environment and Husbandry

Housing

All animals were housed in groups of 3-5 in large cages kept in ventilated shelves under standard animal care conditions, except as described in section 7.2 during DED induction. A refuge, gnawing material, and nesting material were present in every cage.

Lighting

12 hours light/12 hours dark, except when room lights were turned on/off during the normal cycle to accommodate study procedures.

Food

All animals had access to certified rodent diet 5053.

Water

Water was available ad libitum to each animal via an automatic watering device. No contaminants are known to be present in the water at levels that would be expected to have interfered with the results of this study.

Identification

Animals received an ear tag with a 5-digit ID number for tracking and all animal information is stored in a local Microsoft® Access™ database.

Assignment to Study Groups

Mice were randomly assigned to study groups prior to dosing initiation as shown in Table 1.

Methods

Study Design

Baseline: OGD staining and quantification
Days 1-7: Topical instillation of vehicle or test article; QID (Arm 4-5)
Days 4-8: Exposure to desiccating stress (Arms 2-5)
Days 4-7: Topical instillation of positive control; BID (Arm 3)
Day 8: Quantification of corneal permeability; Enucleation of eyes for future histological quantification of conjunctival goblet cell density in each eye and quantification of CD4⁺ T cells in conjunctiva

Induction of Dry Eye Disease (DED)

Dry eye disease was induced in 6-8-week-old, female C57BL/6J mice by exposure to desiccating stress (DS). Animals were acclimated at 55% relative humidity (RH) for one week prior initiation of DS. DS was induced by subcutaneous injection of scopolamine hydrobromide (0.5 mg/0.2 ml; S0929; Sigma-Aldrich, St. Louis, MO) four times per day (8:30 am, 11:00 am, 2:00 pm, and 4:30 pm) throughout the study, except Day 5 when a single injection was given 1 hour prior to OGD staining. Mice were placed in a cage with perforated screens on both sides to allow constant airflow from a fan, and RH was tightly controlled at 20%, and the temperature was maintained at 25° C. in environmental chambers. Animals were maintained on a 12 h light cycle. Control mice were maintained in a nonstressed (NS) environment at 55% RH without exposure to a forced air draft, and the temperature was maintained at 25° C. in a separate chamber.

Bilateral Topical Instillation

Topical formulations of vehicle or test agents were administered to both eyes via a positive displacement pipette in a volume of 3 μl twice-daily (BID) at approximately 8:30 am and 4:30 pm each day. For the procedure, fully awake animals were gently restrained by the scruff of the neck and the eyelids were gently pulled away from the eye by the forefinger and thumb of the individual performing the procedure. Following administration, the animals were held for an additional 60 seconds to allow the delivered agent to fully disperse.

Assessment of Corneal Barrier Function with Oregon Green Dextran (OGD)

Corneal staining was measured by penetration of Oregon Green Dextran (OGD; ThermoFisher; D7172). For baseline assessment, 0.5 μL of OGD was instilled on the cornea of both eyes, and the mouse were housed in the dark for 1 min before washing with BSS and briefly anesthetizing with isoflurane before imaging. For corneal permeability assessment at Day 8, 0.5 μL of OGD was instilled on the cornea of both eyes, and the mouse was immediately housed in the dark for one minute, followed by euthanization and immediate imaging. Eyes were washed with 2 mL of balance saline solution (BSS, Alcon; 0065 0795-50). Excess liquid was blotted from around the eye with filter paper. Digital images were captured, and the mean fluorescence intensity within a 2 mm central corneal ring was measured with NIS Elements software (Nikon).

Tissue Collection

At study termination on Day 8, the left eyes were enucleated with intact conjunctiva and fixed in 10% formalin, and stored at RT. The right eyes were enucleated with intact conjunctiva, suspended in optimal cutting temperature (OCT) compound, and flash frozen in liquid. nitrogen.

Histology and Periodic Acid-Schiff Staining and Conjunctival Goblet-Cells (GC) Measurement

Enucleated mouse eyes with intact conjunctiva were fixed in 10% formalin and embedded in paraffin. Six-micrometer sections were stained with periodic acid-Schiff (PAS) reagent. GC density in the superior and inferior conjunctiva are measured using NIS Elements Basic Research microscope imaging software (version 3.0; Nikon, Melville, NY) using a 10× objective. GC density quantification was performed on Arms 1, 2, 4, and 5 only.

Data Handling

Data Collection and Analysis

Test article information, in-life data, and bioanalytical data were collected as digital files, or on paper as necessary. Data and statistical analysis were performed using Graphpad Prism version 5.00.

Study Deviations

There were no deviations to the study design in the original protocol.

Data and Statistical Analyses

Statistical analyses were performed with GraphPad Prism software (version 5.0) using an unpaired student t-test. Only changes with a p-value<0.05 were deemed statistically significant.

Results

Corneal Permeability

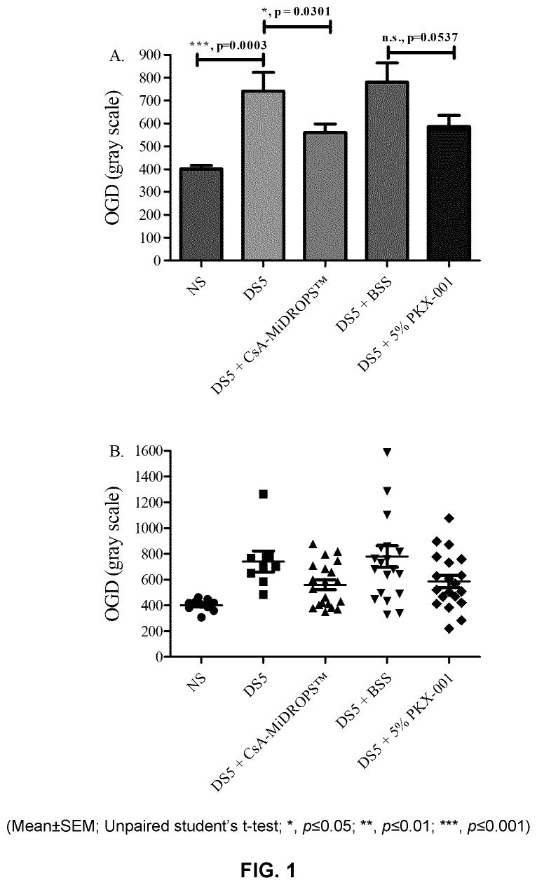

Corneal permeability was measured at Day 8 to assess the efficacy of 5% AAGP™ on corneal barrier function. On Day 8, all study arms exposed to DS showed an increase in mean corneal permeability compared to unexposed mice, and the difference was significant for the mice exposed to DS but left untreated, or administered with the BSS vehicle alone (FIGS. 1A and 1B; Tables 2-6; Unpaired student's t-test). Administration of 0.1% CsA-MiDROPS™ significantly reduced corneal permeability compared to the untreated mice (FIGS. 1A and 1B; Unpaired student's t-test). Administration of BSS had no effect on reducing mean corneal permeability compared to DS-exposed mice left untreated (FIGS. 1A and 1B; Tables 2-6; Unpaired student's t-test). Administration of 5% AAGP™ reduced mean corneal permeability compared to BSS alone, and the difference approached, but did not achieve statistical significance (FIGS. 1A and 1B; Tables 2-6; Unpaired student's t-test).

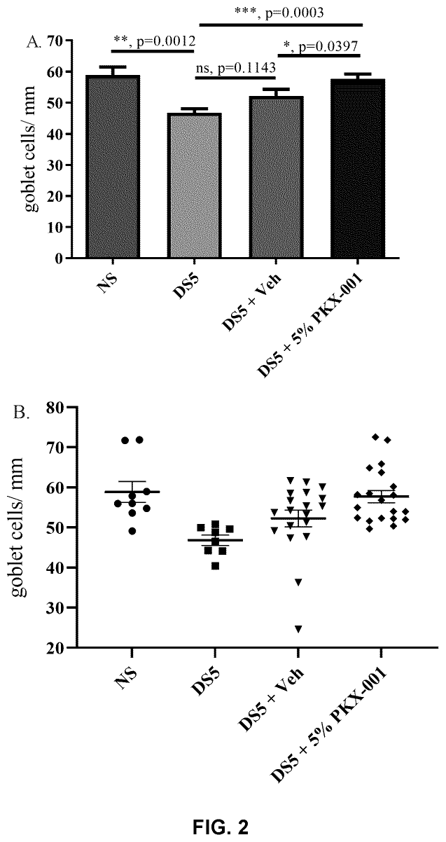

Conjunctival goblet cell density was also measured, and the results show (FIG. 2 left and right) treatment with 5% AAGP™ (PKX-001) prevents the decrease in goblet cell density caused by desiccating stress. GC loss was quantified as a measure of ocular surface injury by terminal histological analysis. The mean GC number was reduced from 58.88 to 46.82 in mice exposed to DS and left untreated compared to non-stressed mice (FIG. 2; **p=0.0012; Unpaired Student's t-test). There was no statistical difference in mean GC number between the vehicle administered and untreated DS-exposed mice (FIG. 2). Mice administered 5% PKX-001 had significantly higher mean GC density compared to both the untreated, and the vehicle administered mice exposed to DS (FIG. 2; Unpaired Student's t-test).

CD4+ T Cell Infiltration

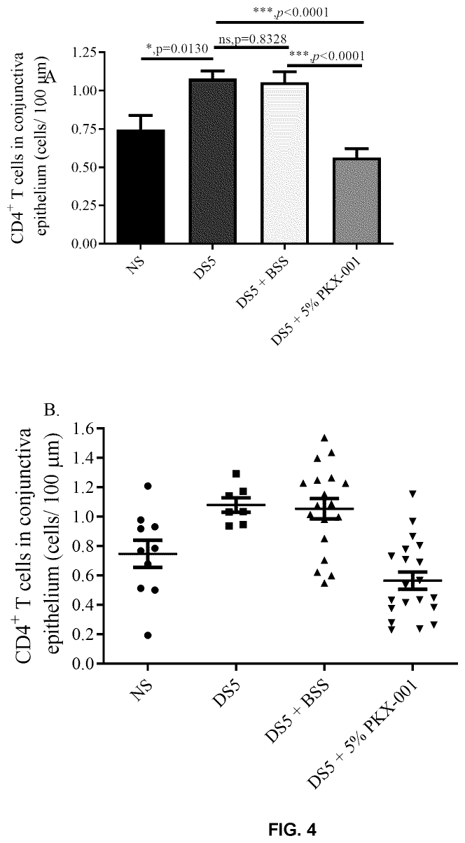

CD4+ T cells infiltration into the conjunctival epithelium is a primary clinical indicator of DED. The presence of CD4+ T cells in the conjunctival epithelium significantly increased in DED-induced mice left untreated (FIG. 4; *p=0.0130; Unpaired Student's t-test). Vehicle administration had no effect on reducing T-cell infiltration, whereas bilateral topical administration of 5% PKX-001 significantly reduced T-cell infiltration in DS-induced mice (FIG. 4 Table 7; ***, p≤0.0001; Unpaired Student's t-test).

TABLE 2
Corneal Permeability Column Statistics

| | | | DS5 + | | |
|---|---|---|---|---|---|
| | | | CsA- | DS5 + | DS5 + 5% |
| | NS | DS5 | MiDROPS | BSS | AAGP ™ |
| Number of | 10 | 8 | 20 | 20 | 20 |
| values | | | | | |
| Minimum | 308.8 | 484.3 | 349.4 | 328.2 | 220.2 |
| 25% Percentile | 376.6 | 601.8 | 407.1 | 491.9 | 433.4 |
| Median | 413.8 | 700.5 | 556.6 | 706.2 | 545.8 |
| 75% Percentile | 433 | 774.2 | 703.1 | 854.7 | 752.8 |
| Maximum | 463.2 | 1265 | 876.2 | 1679 | 1076 |
| Mean | 402.6 | 741.2 | 560.3 | 780.4 | 587.5 |
| Std. Deviation | 45.28 | 232.4 | 169.3 | 376.1 | 215.2 |
| Std. Error | 14.32 | 82.18 | 37.87 | 84.1 | 48.11 |
| Lower 95% | 370.2 | 546.9 | 481.1 | 604.4 | 486.8 |
| CI of mean | | | | | |
| Upper 95% | 434.9 | 935.5 | 639.6 | 956.5 | 688.2 |
| CI of mean | | | | | |
| Sum | 4026 | 5930 | 11207 | 15609 | 11750 |

TABLE 3
OGD - Raw Data

| NS | | | DS5 | | |
|---|---|---|---|---|---|
| | | Mean | | | Mean |
| Animal ID | Eye | Intensity | Animal ID | Eye | Intensity |
| 21230 | L | 382.56 | 21236 | L | 650.12 |
| 21230 | R | 386.88 | 21236 | R | 484.26 |
| 21231 | L | 407.65 | 21237 | L | 776.6 |
| 21231 | R | 463.22 | 21237 | R | 766.91 |
| 21233 | L | 419.93 | 21238 | L | 699.86 |
| 21233 | R | 421.78 | 21238 | R | 701.12 |
| 21234 | L | 308.82 | 21240 | L | 585.75 |
| 21234 | R | 448.09 | 21240 | R | 1265.13 |
| 21235 | L | 427.94 | | | |
| 21235 | R | 358.67 | | | |

| DS5 + CsA-MiDROPS | | | DS5 + BSS | | | DS5 + 5% AAGP ™ | | |
|---|---|---|---|---|---|---|---|---|
| | | Mean | | | Mean | | | Mean |
| Animal ID | Eye | Intensity | Animal ID | Eye | Intensity | Animal ID | Eye | Intensity |
| 21241 | L | 349.39 | 21251 | L | 1587.52 | 21262 | L | 522.49 |
| 21241 | R | 379.19 | 21251 | R | 680.28 | 21262 | R | 778.63 |
| 21242 | L | 383.28 | 21252 | L | 1102.85 | 21262 | L | 412.73 |
| 21242 | R | 584.99 | 21252 | R | 636.84 | 21262 | R | 469.8 |
| 21243 | L | 876.17 | 21253 | L | 497.16 | 21264 | L | 421.74 |
| 21243 | R | 414.46 | 21253 | R | 1679.16 | 21264 | R | 383.5 |
| 21244 | L | 747.49 | 21254 | L | 643.49 | 21265 | L | 896.98 |
| 21244 | R | 684.04 | 21254 | R | 434.26 | 21265 | R | 609.09 |
| 21245 | L | 598.95 | 21255 | L | 339.88 | 21267 | L | 468.34 |
| 21245 | R | 819.4 | 21255 | R | 328.21 | 21267 | R | 872.1 |
| 21246 | L | 797.22 | 21256 | L | 1286.6 | 21268 | L | 284.06 |
| 21246 | R | 404.64 | 21256 | R | 762.92 | 21268 | R | 509.19 |
| 21247 | L | 528.29 | 21257 | L | 446.98 | 21269 | L | 569.19 |
| 21247 | R | 584.89 | 21257 | R | 490.13 | 21269 | R | 632.65 |
| 21248 | L | 369.44 | 21259 | L | 847.43 | 21270 | L | 1076.4 |
| 21248 | R | 421.98 | 21259 | R | 731.84 | 21270 | R | 759.34 |
| 21249 | L | 658.84 | 21260 | L | 758.2 | 21271 | L | 220.15 |
| 21249 | R | 430.71 | 21260 | R | 680.5 | 21271 | R | 503.19 |
| 21250 | L | 463.86 | 21261 | L | 817.26 | 21272 | L | 733.05 |
| 21250 | R | 709.47 | 21261 | R | 857.17 | 21272 | R | 627.44 |
| ave | | 560.335 | | | | ave | | 587.503 |

TABLE 4
OGD - Statistics
NS vs DS5

| | | |
|---|---|---|
| Unpaired t test | | |
| P value | | 0.0003 |
| P value summary | *** | |
| Are means signif. different? (P < 0.05) | Yes | |
| One- or two-tailed P value? | Two-tailed | |
| t, df | t = 4.534 df = 16 | |
| How big is the difference? | | |
| Mean ± SEM of column A | 402.6 ± 14.32 N = 10 | |
| Mean ± SEM of column B | 741.2 ± 82.18 N = 8 | |
| Difference between means | −338.7 ± 74.69 | |
| 95% confidence interval | −497.0 to −180.3 | |
| R squared | | 0.5624 |
| F test to compare variances | 26.35, 7, 9 | |
| F, DFn, Dfd | | |
| P value | P < 0.0001 | |
| P value summary | *** | |
| Are variances significantly different? | Yes | |

DS5 vs DS5 + CsA-MiDROPS

| | | |
|---|---|---|
| Unpaired t test | | 0.0301 |
| P value | | |
| P value summary | * | |
| Are means signif. different? (P < 0.05) | Yes | |
| One- or two-tailed P value? | Two-tailed | |
| t, df | t = 2.295 df = 26 | |
| How big is the difference? | | |
| Mean ± SEM of column B | 741.2 ± 82.18 N = 8 | |
| Mean ± SEM of column C | 560.3 ± 37.87 N = 20 | |
| Difference between means | 180.9 ± 78.82 | |
| 95% confidence interval | 18.83 to 342.9 | |
| R squared | | 0.1684 |
| F test to compare variances | | |
| F, DFn, Dfd | 1.884, 7, 19 | |
| P value | | 0.2577 |
| P value summary | ns | |
| Are variances significantly different? | No | |

TABLE 5
OGD - Statistics
DS5 + BSS vs DS5 + 5% AAGP ™

| | | |
|---|---|---|
| Unpaired t test | | |
| P value | | 0.0537 |
| P value summary | ns | |
| Are means signif. different? (P < 0.05) | No | |
| One- or two-tailed P value? | Two-tailed | |
| t, df | t = 1.991 df = 38 | |
| How big is the difference? | | |
| Mean ± SEM of column D | 780.4 ± 84.10 N = 20 | |
| Mean ± SEM of column E | 587.5 ± 48.11 N = 20 | |
| Difference between means | 192.9 ± 96.89 | |
| 95% confidence interval | −3.291 to 389.2 | |
| R squared | | 0.09448 |
| F test to compare variances | 3.055, 19, 19 | |
| F, DFn, Dfd | | |
| P value | | 0.019 |
| P value summary | * | |
| Are variances significantly different? | Yes | |

DS5 vs DS5 + 5% AAGP ™

| | | |
|---|---|---|
| Unpaired t test | | |
| P value | | 0.1068 |
| P value summary | ns | |
| Are means signif. different? (P < 0.05) | No | |
| One- or two-tailed P value? | Two-tailed | |
| t, df | t = 1.671 df = 26 | |
| How big is the difference? | | |
| Mean ± SEM of column B | 741.2 ± 82.18 N = 8 | |
| Mean ± SEM of column E | 587.5 ± 48.11 N = 20 | |
| Difference between means | 153.7 ± 92.01 | |
| 95% confidence interval | −35.46 to 342.9 | |
| R squared | | 0.09694 |
| F test to compare variances | 1.167, 7, 19 | |
| F, DFn, Dfd | | |
| P value | | 0.7324 |
| P value summary | ns | |
| Are variances significantly different? | No | |

TABLE 6
OGD - Statistics
DS5 vs DS5 + BSS

| | | |
|---|---|---|
| Unpaired t test | | |
| P value | | 0.787 |
| P value summary | ns | |
| Are means signif. | No | |
| different? (P < 0.05) | | |
| One- or two-tailed P value? | Two-tailed | |
| t, df | t = 0.2730 df = 26 | |
| How big is the difference? | | |
| Mean ± SEM of column B | 741.2 ± 82.18 N = 8 | |
| Mean ± SEM of column D | 780.4 ± 84.10 N = 20 | |
| Difference between means | −39.22 ± 143.7 | |
| 95% confidence interval | −334.6 to 256.1 | |
| R squared | | 0.002858 |
| F test to compare variances | 2.618, 19, 7 | |
| F, DFn, Dfd | | |
| P value | | 0.1969 |
| P value summary | ns | |
| Are variances | No | |
| significantly different? | | |

DS5 vs DS5 + BSS

| | | |
|---|---|---|
| Unpaired t test | | |
| P value | | 0.004 |
| P value summary | ** | |
| Are means signif. | Yes | |
| different? (P < 0.05) | | |
| One- or two-tailed P value? | Two-tailed | |
| t, df | t = 3.138 df = 28 | |
| How big is the difference? | | |
| Mean ± SEM of column A | 402.6 ± 14.32 N = 10 | |
| Mean ± SEM of column D | 780.4 ± 84.10 N = 20 | |
| Difference between means | −377.9 ± 120.4 | |
| 95% confidence interval | −624.5 to −131.3 | |
| R squared | | 0.2602 |
| F test to compare variances | 68.99, 19, 9 | |
| F, DFn, Dfd | | |
| P value | P < 0.0001 | |
| P value summary | *** | |
| Are variances | Yes | |
| significantly different? | | |

TABLE 7
T-Cell Density Column Statistics

| | | | DS5 + | DS5 + 5% |
|---|---|---|---|---|
| | NS | DS5 | BSS | PKX-001 |
| Number of values | 10 | 7 | 18 | 20 |
| Minimum | 0.1922 | 0.9365 | 0.5482 | 0.2307 |
| 25% Percentile | 0.5093 | 0.9462 | 0.8143 | 0.3767 |
| Median | 0.7759 | 1.034 | 1.082 | 0.4907 |
| 75% Percentile | 0.9429 | 1.172 | 1.253 | 0.7663 |
| Maximum | 1.208 | 1.293 | 1.538 | 1.152 |
| Mean | 0.747 | 1.079 | 1.054 | 0.5644 |
| Std. Deviation | 0.2905 | 0.1295 | 0.2946 | 0.2614 |
| Std. Error of Mean | 0.09186 | 0.04895 | 0.06944 | 0.05845 |
| Lower 95% CI of mean | 0.5392 | 0.9595 | 0.9079 | 0.4421 |
| Upper 95% CI of mean | 0.9548 | 1.199 | 1.201 | 0.6868 |
| Sum | 7.47 | 7.555 | 18.98 | 11.29 |

The results of the CD4+ T cells infiltration assay show that unexpectedly, in addition to providing protection against a hostile stimulus, AAGP™ suppresses T-cell response in addition to reducing the ocular inflammatory response.

### Example 2

### An Efficacy Evaluation of Two Different Doses of AAGP™ in an Acute Mouse Model of Dry Eye Disease

The objective of this study was to investigate the dose-range of AAGP™ by comparing the efficacy of topical administration of 2% AAGP™, or 5% AAGP™, in an acute mouse model of DED. Another objective was to compare the potential therapeutic effect of twice a day dosing, or four times a day dosing of AAGP™ in an acute mouse model of DED. Another objective was to evaluate the efficacy of AAGP™ administered daily for three days prior to and four days post-induction of DED, to administration of AAGP™ daily for four days post-induction of DED.

### LIST OF ABBREVIATIONS

BID Two times a day
BSS Balanced salt solution
DED Dry Eye Disease
DS Desiccating stress
OCT Optimal cutting temperature
OGD Oregon Green Dextran
NS Non-stressed
QID Four times a day
RH Relative humidity
RT Room temperature
SOP Standard Operating Procedure

Methods:

Age and weight matched female C57BL/6J mice were divided into eight separate arms as shown in Table 8. On Days 4-8, the mice in study arms 2 through 8 were also kept in adverse environmental chambers (low humidity and constant airflow from a fan), and exposed to desiccating stress (DS) by subcutaneous injections of scopolamine hydrobromide 4 times a day at 8:30 AM, 11:00 AM, 2:00 PM, and 4:30 PM, on Days 4 through 7, and once on Day 8 at 8:30 AM, to expose the animals to DS and induce acute DED. Treatment duration and frequency varied across the study arms. Mice in Arm 2 were left untreated. Mice in Arm 3 received twice a day bilateral topical administration of the positive control, 0.1% CsA-MiDROPS™ (Coursey et al. Once-Daily Cyclosporine-A-MiDROPS™ for Treatment of Dry Eye Disease Translational Vision Science & Technology 2018; 7:1-13), on Days 4-7. The mice in Arm 4 received twice a day bilateral topical administration of 5% AAGP™ on Days 1-7. The mice in Arm 5 received four times a day bilateral topical administration of the vehicle, BSS, on Days 1-7. The mice in Arm 6 received four times a day bilateral topical administration of 5% AAGP™ on Days 4-7. The mice in Arm 7 and Arm 8 received either 2% AAGP™ (Arm 7), or 5% AAGP™ (Arm 8), four times a day via bilateral topical administration on Days 1-7.

Corneal permeability was assessed by Oregon Green Dextran (OGD) staining at baseline just prior to enrollment into study arms to determine if any mice met exclusion criteria. Corneal permeability was again measured on Day 8 for efficacy comparisons between mice in all study arms.

TABLE 8
Arm Allocation

| Arm | Induction | Treatment | Treatment Details | Assessment | Animal ID |
|---|---|---|---|---|---|
| 1 | None | None | n/a | Quantification of | 22196, 22197, 22198, |
| | | n = 6 | | corneal permeability | 22234, 22235, 22236 |
| 2 | Desiccating | None | n/a | in all eyes at Day 8; | 22199, 22200, 22201, |
| | Stress on | n = 5 | | Enucleation and | 22237, 22238 |
| 3 | Days 4-8 | 0.1% CsA- | Bilateral topical | collection of eyes for | 22202, 22203, 22204, |
| | | MIDROPS ™ | instillation, BID on | histological | 22240, 22241, 22242 |
| | | n = 6 | Days 4-7 | examination on Day 8 | |
| | | | (2 μl/instillation) | | |
| 4 | | 5% AAGP ™ | Bilateral topical | | 22205, 22206, 22208, |
| | | n = 10 | instillation, BID on | | 22209, 22211, 22243, |
| | | | Days 1-7 | | 22244, 22245, 22246, |
| | | | (3 μl/instillation) | | 22247 |
| 5 | | Vehicle (BSS) | Bilateral topical | | 22212, 22213, 22215, |
| | | n = 8 | instillation, QID, on | | 22218, 22249, 22250, |
| | | | Days 1-7 | | 22252, 22253 |
| | | | (3 μl/instillation) | | |
| 6 | | 5% AAGP ™ | Bilateral topical | | 22219, 22220, 22221, |
| | | n = 10 | instillation, QID on | | 22222, 22223, 22255, |
| | | | Days 4-7 | | 22256, 22257, 22258, |
| | | | (3 μl/instillation) | | 22259 |
| 7 | | 2% AAGP ™ | Bilateral topical | | 22224, 22225, 22226, |
| | | n = 9 | instillation, QID, on | | 22227, 22228, 22260, |
| | | | Days 1-7 | | 22262, 22263, 22264 |
| 8 | | 5% AAGP ™ | (3 μl/instillation) | | 22229, 22230, 22231, |
| | | n = 10 | | | 22232, 22233, 22265, |
| | | | | | 22266, 22267, 22269, |
| | | | | | 22270 |

Study Objective

Efficacy Assessment

To determine the timing of effect of AAGP™, formulated at 2% or 5%, to reduce corneal permeability after repeat topical instillation in an acute mouse model of dry eye disease induced by desiccating stress and scopolamine.

Test Materials and Equipment

Test Article(s) Preparation and Storage

AAGP™ was provided as a powder of ˜500 mg and stored at −20° C. upon arrival. The test article was kept protected from light throughout the study. AAGP™ was formulated in BSS and aliquoted into daily use aliquots as follows:
1) AAGP™ powder was removed from −20° C., placed in a desiccating chamber, and brought to room temperature for ˜30 minutes.
2) The required amount of AAGP™ was weighed and into an appropriate container using a potency value of 87.79% (AAGP™ Lot number PD150709-1R).
3) 80% of the final required volume of vehicle (BSS sterile irrigating solution, Alcon, cat #0065079550) was added.
4) The solution was stirred until complete dissolution of AAGP™
5) The pH was measured and adjusted to 5-8 by slowly pipetting 1 N NaOH solution. The initial and adjusted pH values were recorded as shown in Table 9.
6) The AAGP™ formulation was transferred into a volumetric flask and vehicle was added to the required final volume, to achieve the correct final concentration.
7) The osmolarity and viscosity of the final solution were measured and recorded as shown in Table 9.
8) The solution was sterilized by filtration through a 0.22 μm GV (PVDF) filter (Millipore) into a sterile vial and aliquoted into daily use vials for administration.
9) The formulations were stored at 4° C.±2° C. until the day of administration. On the day of administration, the formulations were warmed to room temperature prior to use.

TABLE 9
AAGP ™ Formulation Quality Control Records

| | | Initial | Adjusted | | |
|---|---|---|---|---|---|
| | Date | pH | pH | Osmolarity | Viscosity |
| 5% AAGP ™ | Jun. 19, | 5.37 | n/a | 409 mOsm/kg | 0.0 cP |
| | 2020 | | | | |
| 2% AAGP ™ | Jun. 19, | 5.42 | n/a | 342 mOsm/kg | 0.0 cP |
| | 2020 | | | | |
| 5% AAGP ™ | Jun. 19, | 5.38 | n/a | 408 mOsm/kg | 0.0 cP |
| | 2020 | | | | |
| 2% AAGP ™ | Jun. 19, | 5.90 | n/a | 345 mOsm/kg | 0.0 cP |
| | 2020 | | | | |

Test System

Species, Strain, Sex, Age, Weight, Supplier
Species: Mouse
Strain: C57BL/6J
Sex: Female
Age Range: 6 weeks old at time of arrival to test facility
Weight: >15-25 g
Supplier: Jackson Laboratories
Number of Study Animals: 64
Number of Spare Animals 0

Environment and Husbandry

Housing

All animals were housed in groups of 3-5 in large cages kept in ventilated shelves under standard animal care conditions, except as described in section 7.2 during DED induction. A refuge, gnawing material, and nesting material were present in every cage.

Lighting

12 hours light/12 hours dark, except when room lights were turned on/off during the normal cycle to accommodate study procedures.

Food

All animals had access to certified rodent diet 5053.

Water

Water was available ad libitum to each animal via an automatic watering device. No contaminants are known to be present in the water at levels that would be expected to have interfered with the results of this study.

Identification

Animals received an ear tag with a 5-digit ID number for tracking and all animal information is stored in a local MS Access database.

Assignment to Study Groups

Mice were randomly assigned to study groups prior to dosing initiation as shown in Table 1.

Methods

Study Design
Baseline: Oregon Green Dextran (OGD) staining and quantification
Days 1-7: Topical instillation of Vehicle (3 μl volume), QID (Arm 5), or test article (3 μl volume), QID (Arm 7, 8) or BID (Arm 4)
Days 4-8: Exposure to desiccating stress (Arms 2-8)
Days 4-7: Topical instillation of positive control (2 μl/eye), BID (Arm 3)
Days 4-7: Topical instillation of vehicle or test agent (3 μl/eye), QID (Arm 6).
Day 8: Quantification of corneal permeability; Enucleation of eyes for future histological study

Induction of Dry Eye Disease (DED)

Dry eye disease was induced in 6-8-week-old, female C57BL/6J mice by exposure to desiccating stress (DS). Animals were acclimated at 55% relative humidity (RH) for one-week prior initiation of DS. DS was induced by subcutaneous injection of scopolamine hydrobromide (0.5 mg/0.2 ml; S0929; Sigma-Aldrich, St. Louis, MO) four times per day (8:30 am, 11:00 am, 2:00 pm, and 4:30 pm) throughout the study, except Day 8 when a single injection was given 1 hour prior to OGD staining. Mice were placed in a cage with perforated screens on both sides to allow constant airflow from a fan, and RH was tightly controlled at 20%, and the temperature was maintained at 25° C. in environmental chambers. Animals were maintained on a 12 h light cycle. Control mice were maintained in a non-stressed (NS) environment at 55% RH without exposure to a forced air draft, and the temperature was maintained at 25° C. in a separate chamber.

Bilateral Topical Instillation

Topical formulations of vehicle (BSS) or AAGP™ were administered to both eyes via a positive displacement pipette in a volume of 3 μl/instillation four times per day (QID) at approximately 8:30 am, 11:00 am, 2:00 pm and 4:30 pm each day on Days 1 through 7 to Arms 5, 7, and 8, or on days 4-7 to Arm 6. In Arm 4, AAGP™ was administered in a similar manner but only two times per day (BID) at approximately 8:30 am and 4:30 pm, Days 1 through 7. Topical formulations of the positive control (0.1% CsA-MiDROPS™) were administered in a volume of 2 μl/instillation two times per day (BID) at approximately 8:30 am and 4:30 pm each day on Days 4 through 7. For the procedure, fully awake animals were gently restrained by the scruff of the neck and the eyelids were gently pulled away from the eye by the forefinger and thumb of the individual performing the procedure. Following administration, the animals were held for an additional 60 seconds to allow the delivered agent to fully disperse.

Assessment of Corneal Barrier Function with Oregon Green Dextran (OGD)

Corneal staining was measured by penetration of Oregon Green Dextran (OGD; ThermoFisher; D7172). For baseline assessment, 0.5 μL of OGD was instilled on the cornea of both eyes, and the mouse were housed in the dark for 1 min before washing with BSS and briefly anesthetizing with isoflurane before imaging. For corneal permeability assessment at Day 8, 0.5 μL of OGD was instilled on the cornea of both eyes, and the mouse was immediately housed in the dark for one minute, followed by euthanization and immediate imaging. Eyes were washed with 2 mL of balance saline solution (BSS, Alcon; 0065 0795-50). Excess liquid was blotted from around the eye with filter paper. Digital images were captured, and the mean fluorescence intensity within a 2 mm central corneal ring was measured with NIS Elements software (Nikon).

Tissue Collection

At study termination on Day 8, the left eyes were enucleated with intact conjunctiva and fixed in 10% formalin, and stored at RT. The right eyes were enucleated with intact conjunctiva, suspended in optimal cutting temperature (OCT) compound, and flash frozen in liquid nitrogen.

Data Handling

Data Collection and Analysis

Test article information, in-life data, and bioanalytical data were collected as digital files, or on paper as necessary. All information is stored at the EyeCRO test facility. Data and statistical analysis were performed using GraphPad Prism version 5.00.

Data and Statistical Analyses

Statistical analyses were performed with GraphPad Prism software (version 8.4.3) using an unpaired student t-test. Only changes with a p-value<0.05 were deemed statistically significant.

### RESULTS

Corneal permeability was measured at Day 8 to assess: 1) the efficacy AAGP™ compared to vehicle control, 2) the efficacy of 2% AAGP™ compared to 5% AAGP™, 3) the efficacy of pretreatment with AAGP™ prior to induction of DED compared to treatment commencing upon DED induction, and, 4) the efficacy of twice a day dosing compared to four times a day.

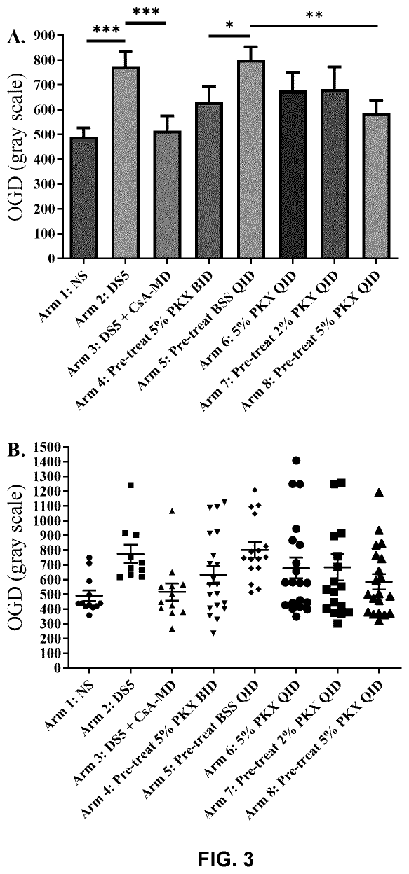

In this study, there was a significant induction of DED as indicated by significantly greater corneal permeability in untreated DS-exposed mice compared to non-stressed mice (FIGS. 2A-B; ***, p=0.0006; Mann-Whitney t-test). Vehicle (BSS) treatment had no protective effect on corneal permeability (p>0.05 vs. DS control). Each of the AAGP™ treatment arms showed reduced mean corneal permeability compared to the untreated, or vehicle treated arms (FIG. 3A-B; Table 10). Pretreatment with 5% AAGP™, QID or BID was characterized by significantly reduced mean corneal permeability compared to the BSS control (FIG. 3A-B; Table 10; *, p≤0.05; **p<0.01; Mann-Whitney t-test). Administration of 2% AAGP™ QID (with pretreatment) or 5% AAGP™ QID (without pretreatment) was also associated with reduced mean corneal permeability compared to BSS, but the difference was not statistically significant. Positive control (0.1% CsA-MiDROPS™) significantly reduced corneal permeability as compared to the control DS group (***p≤0.001; Mann-Whitney t-test).

TABLE 10
Corneal Permeability Column Statistics

| | | | | Arm 4: | | | Arm 7: | Arm 8: |
|---|---|---|---|---|---|---|---|---|
| | | | Arm 3: | Pre-treat | Arm 5: | Arm 6: | Pre-treat | Pre-treat |
| | Arm 1: | Arm 2: | DS5 + | 5% PKX | Pre-treat | 5% PKX | 2% PKX | 5% PKX |
| | NS | DS5 | CsA-MD | BID | BSS QID | QID | QID | QID |
| Number of values | 12 | 10 | 12 | 20 | 16 | 20 | 18 | 20 |
| Minimum | 357.6 | 615.2 | 266.6 | 234.4 | 513 | 348.1 | 301.2 | 317.9 |
| 25% Percentile | 418.8 | 630 | 386.1 | 409.6 | 677.4 | 430.5 | 396.4 | 370.9 |
| Median | 437.8 | 697.9 | 475.2 | 565 | 749.5 | 579 | 542.5 | 499.5 |
| 75% Percentile | 565.2 | 906.4 | 563.6 | 874.5 | 991.3 | 859 | 900.3 | 756.4 |
| Maximum | 750 | 1241 | 1066 | 1125 | 1208 | 1409 | 1653 | 1189 |
| Range | 392.4 | 626 | 799.7 | 890.5 | 695 | 1061 | 1352 | 871.4 |
| Mean | 491.1 | 774.3 | 515.7 | 631.1 | 800.8 | 678.8 | 683.1 | 585.5 |
| Std. Deviation | 125 | 196.7 | 202.9 | 272.3 | 209.2 | 317.3 | 376.2 | 233.8 |
| Std. Error of Mean | 36.08 | 62.19 | 58.56 | 60.9 | 52.3 | 70.94 | 88.67 | 52.29 |
| Lower 95% CI | 411.6 | 633.6 | 386.8 | 503.6 | 689.3 | 530.3 | 496 | 476.1 |
| of mean | | | | | | | | |
| Upper 95% CI | 570.5 | 915 | 644.6 | 758.5 | 912.3 | 827.2 | 870.1 | 695 |
| of mean | | | | | | | | |

Table 11 shows the percentage of reduction in corneal permeability for each treatment group compared to the BSS vehicle treatment. In this table, permeability values were normalized to the background (no DS) level, with % reduction showing to what degree the elevated corneal permeability returns back to normal (background) levels in each treatment arm. Administration of 5% and 2% AAGP™, QID (with pretreatment) was characterized by 69.5% and 38.0% reduction of elevated permeability observed in the BSS group. Less frequent (BID) administration of 5% AAGP™ (with pretreatment) was associated with 54.8% reduction of elevated permeability. Without pretreatment, 5% AAGP™ QID administration led to 39.4% reduction. Positive control 0.1% CsA-MiDROPS™ inhibited elevation of corneal permeability by 91.3% (as compared to no-treatment DS5 group).

TABLE 11
Percent Reduction in DS-induced Corneal Permeability.

| | Mean | Increase | % Reduction |
|---|---|---|---|
| Treatment Group | Intensity | over NS | vs. BSS* |
| NS | 491.07 | | |
| DS5 | 774.28 | 283.22 | |
| CSA-MIDROPS ™ | 515.71 | 24.64 | 91.3* |
| Pre-treat 5% PKX BID | 631.06 | 139.99 | 54.8 |
| BSS QID | 800.79 | 309.72 | |
| 5% PKX QID | 678.76 | 187.69 | 39.4 |
| Pre-treat 2% PKX QID | 683.06 | 191.99 | 38.0 |
| Pre-treat 5% PKX QID | 585.53 | 94.46 | 69.5 |

*% reduction (for values normalized to background NS corneal permeability) is calculated against BSS control group except for CsA-MiDROPS ™ where % reduction was determined vs. Control DS5 group.

When comparing different treatment regiments, the following observations can be made:

Delivery of AAGP™ at 5% four times a day for seven days resulted in lower mean corneal permeability compared to delivery of AAGP™ at 2% four times a day for seven days (Group mean permeability of 585.5 and 683.1, respectively), but the difference was not statistically significant (Tables 9 and 12).

5% AAGP™, QID administration with a 3-day pretreatment period resulted in lower mean corneal permeability as compared to 5% AAGP™ QID administration without pretreatment (Group mean permeability of 585.5 and 678.8, respectively), but the difference was not statistically significant (Tables 9 and 12).

Mean corneal permeability was also lower in mice administered 5% AAGP™ four times a day for seven days compared to mice administered 5% AAGP™ twice a day for seven days (Mean group permeability of 585.5 and 631.1, respectively), but the difference was not statistically significant (Tables 9 and 12).

Mice exposed to DS but left untreated showed a significant induction of DED compared to non-stressed mice as measured by OGD staining of corneal permeability. Administration of the BSS vehicle had no effect on reduction of corneal permeability compared to untreated DS-exposed mice. Administration of 5% AAGP™ either twice a day, or four times a day, for seven days, significantly reduced the DS-induced corneal permeability compared to the vehicle administration. Administration of 2% AAGP™ four times a day for seven days, or 5% AAGP™ four times a day for four days reduced corneal permeability compared to vehicle, but the differences were not statistically significant. There was no statistical difference in mean corneal permeability across the AAGP™ treatment arms.

In a mouse model of DED, 5% AAGP™ significantly reduces corneal permeability following twice a day, or four times a day, topical administration when treatment begins three days prior to disease induction and continues for four days following disease induction.

TABLE 11
OGD - Raw Data
Arm 1: NS

| | | |
|---|---|---|
| 22196 | L | 427.16 |
| 22196 | R | 418.46 |
| 22197 | L | 438.75 |
| 22197 | R | 437.13 |
| 22198 | L | 438.37 |
| 22198 | R | 411.61 |
| 22234 | L | 710.74 |
| 22234 | R | 749.98 |
| 22235 | L | 494.56 |
| 22235 | R | 419.7 |
| 22236 | L | 588.8 |
| 22236 | R | 357.55 |

Arm 2: DS5

| | | |
|---|---|---|
| 22199 | L | 620.27 |
| 22199 | R | 665.12 |
| 22200 | L | 716.64 |
| 22200 | R | 1241.17 |
| 22201 | L | 679.11 |
| 22201 | R | 615.19 |
| 22237 | L | 633.19 |
| 22237 | R | 753.82 |
| 22238 | L | 914.77 |
| 22238 | R | 903.55 |

Arm 3: DS5 + CsA-MD

| | | |
|---|---|---|
| 22202 | L | 552.19 |
| 22202 | R | 566.45 |
| 22203 | L | 554.99 |
| 22203 | R | 1066.24 |
| 22204 | L | 503.37 |
| 22204 | R | 404.43 |
| 22240 | L | 424.49 |
| 22240 | R | 373.34 |
| 22241 | L | 266.59 |
| 22241 | R | 379.96 |
| 22242 | L | 447.12 |
| 22242 | R | 649.31 |

Arm 4: Pre-treat 5% PKX BID

| | | |
|---|---|---|
| 22205 | L | 762.28 |
| 22205 | R | 1090.84 |
| 22206 | L | 603.44 |
| 22206 | R | 1124.8 |
| 22208 | L | 405.32 |
| 22208 | R | 1087.71 |
| 22209 | L | 706.19 |
| 22209 | R | 920.89 |
| 22211 | L | 352.84 |
| 22211 | R | 563.95 |
| 22243 | L | 701.33 |
| 22243 | R | 566.13 |
| 22244 | L | 328.67 |
| 22244 | R | 234.35 |
| 22245 | L | 495.64 |
| 22245 | R | 422.55 |
| 22246 | L | 400.89 |
| 22246 | R | 502.23 |
| 22247 | L | 439.25 |
| 22247 | R | 911.92 |

Arm 5: Pre-treat BSS QID

| | | |
|---|---|---|
| 22212 | L | 1207.97 |
| 22212 | R | 1094.01 |
| 22213 | L | 535.52 |
| 22213 | R | 565.73 |
| 22215 | L | 513 |
| 22215 | R | 740.04 |
| 22218 | L | 676.69 |
| 22218 | R | 1046.26 |
| 22249 | L | 1104.38 |
| 22249 | R | 679.5 |
| 22250 | L | 753.07 |
| 22250 | R | 738.37 |
| 22253 | L | 796.39 |
| 22253 | R | 789.43 |
| 22254 | L | 745.9 |
| 22254 | R | 826.33 |

Arm 6: 5% PKX QID

| | | |
|---|---|---|
| 22219 | L | 1408.75 |
| 22219 | R | 835.7 |
| 22220 | L | 1248.01 |
| 22220 | R | 1249.71 |
| 22221 | L | 657.22 |
| 22221 | R | 945.1 |
| 22222 | L | 397.09 |
| 22222 | R | 577.86 |
| 22223 | L | 402.21 |
| 22223 | R | 439.98 |
| 22255 | L | 459.44 |
| 22255 | R | 866.82 |
| 22256 | L | 581.98 |
| 22256 | R | 580.18 |
| 22257 | L | 415.18 |
| 22257 | R | 427.3 |
| 22258 | L | 445.15 |
| 22258 | R | 577.46 |
| 22259 | L | 711.99 |
| 22259 | R | 348.11 |

Arm 7: Pre-treat 2% PKX QID

| | | |
|---|---|---|
| 22224 | L | 553.23 |
| 22224 | R | 301.21 |
| 22225 | L | 374.86 |
| 22225 | R | 913.95 |
| 22226 | L | 422.27 |
| 22226 | R | 378.25 |
| 22227 | L | 1248.43 |
| 22227 | R | 895.69 |
| 22228 | L | 445.3 |
| 22228 | R | 758.09 |
| 22260 | L | 517.24 |
| 22260 | R | 531.83 |
| 22262 | L | 1653.4 |
| 22262 | R | 1256.6 |
| 22263 | L | 370.05 |
| 22263 | R | 402.46 |
| 22264 | L | 686.81 |
| 22264 | R | 585.42 |

Arm 8: Pre-treat 5% PKX QID

| | | |
|---|---|---|
| 22229 | L | 483.86 |
| 22229 | R | 739.65 |
| 22230 | L | 358.58 |
| 22230 | R | 317.93 |
| 22231 | L | 591.92 |
| 22231 | R | 497.66 |
| 22232 | L | 366.46 |
| 22232 | R | 656.9 |
| 22233 | L | 470.17 |
| 22233 | R | 603.28 |
| 22265 | L | 454.43 |
| 22265 | R | 762.01 |
| 22266 | L | 377.59 |
| 22266 | R | 501.32 |
| 22267 | L | 1189.37 |
| 22267 | R | 931.69 |
| 22269 | L | 831.52 |
| 22269 | R | 844.49 |
| 22270 | L | 362.99 |
| 22270 | R | 368.68 |

TABLE 12
OGD - Statistics

| Column A | Arm 1: NS |
|---|---|
| vs. | vs. |
| Column B | Arm 2: DS5 |
| Mann Whitney test | |
| P value | 0.0006 |
| Exact or approximate P value? | Exact |
| P value summary | *** |
| Significantly different (P < 0.05)? | Yes |
| One- or two-tailed P value? | Two-tailed |
| Sum of ranks in column A, B | 89, 164 |
| Mann-Whitney U | 11 |
| Difference between medians | |
| Median of column A | 437.8, n = 12 |
| Median of column B | 697.9, n = 10 |
| Difference: Actual | −260.1 |
| Difference: Hodges-Lehmann | −246 |
| Column B | Arm 2: DS5 |
| vs. | vs. |
| Column C | Arm 3: DS5 + CsA-MD |
| Mann Whitney test | |
| P value | 0.0008 |
| Exact or approximate P value? | Exact |
| P value summary | *** |
| Significantly different (P < 0.05)? | Yes |
| One- or two-tailed P value? | Two-tailed |
| Sum of ranks in column B, C | 163, 90 |
| Mann-Whitney U | 12 |
| Difference between medians | |
| Median of column B | 697.9, n = 10 |
| Median of column C | 475.2, n = 12 |
| Difference: Actual | 222.6 |
| Difference: Hodges-Lehmann | 248.7 |

TABLE 13
OGD - Statistics

| Column D | Arm 4: Pre-treat 5% PKX BID |
|---|---|
| vs. | vs. |
| Column E | Arm 5: Pre-treat BSS QID |
| Mann Whitney test | |
| P value | 0.0304 |
| Exact or approximate P value? | Exact |
| P value summary | * |
| Significantly different (P < 0.05)? | Yes |
| One- or two-tailed P value? | Two-tailed |
| Sum of ranks in column D, E | 302, 364 |
| Mann-Whitney U | 92 |
| Difference between medians | |
| Median of column D | 565.0, n = 20 |
| Median of column E | 749.5, n = 16 |
| Difference: Actual | −184.4 |
| Difference: Hodges-Lehmann | −183.1 |
| Column E | Arm 5: Pre-treat BSS QID |
| vs. | vs. |
| Column F | Arm 6: 5% PKX QID |
| Mann Whitney test | |
| P value | 0.0771 |
| Exact or approximate P value? | Exact |
| P value summary | ns |
| Significantly different (P < 0.05)? | No |
| One- or two-tailed P value? | Two-tailed |
| Sum of ranks in column E, F | 352, 314 |
| Mann-Whitney U | 104 |
| Difference between medians | |
| Median of column E | 749.5, n = 16 |
| Median of column F | 579.0, n = 20 |
| Difference: Actual | 170.5 |
| Difference: Hodges-Lehmann | 165.3 |

TABLE 14
OGD - Statistics

| Column E | Arm 5: Pre-treat BSS QID |
|---|---|
| vs. | vs. |
| Column G | Arm 7: Pre-treat 2% PKX QID |
| Mann Whitney test | |
| P value | 0.0814 |
| Exact or approximate P value? | Exact |
| P value summary | ns |
| Significantly different (P < 0.05)? | No |
| One- or two-tailed P value? | Two-tailed |
| Sum of ranks in column E, G | 331, 264 |
| Mann-Whitney U | 93 |
| Difference between medians | |
| Median of column E | 749.5, n = 16 |
| Median of column G | 542.5, n = 18 |
| Difference: Actual | 207 |
| Difference: Hodges-Lehmann | 194.2 |
| Column E | Arm 5: Pre-treat BSS QID |
| vs. | vs. |
| Column H | Arm 8: Pre-treat 5% PKX QID |
| Mann Whitney test | |
| P value | 0.006 |
| Exact or approximate P value? | Exact |
| P value summary | ** |
| Significantly different (P < 0.05)? | Yes |
| One- or two-tailed P value? | Two-tailed |
| Sum of ranks in column E, H | 381, 285 |
| Mann-Whitney U | 75 |
| Difference between medians | |
| Median of column E | 749.5, n = 16 |
| Median of column H | 499.5, n = 20 |
| Difference: Actual | 250 |
| Difference: Hodges-Lehmann | 237.9 |

TABLE 15
OGD - Statistics

| Column B | Arm 2: DS5 |
|---|---|
| vs. | vs. |
| Column E | Arm 5: Pre-treat BSS QID |
| Mann Whitney test | |
| P value | 0.6227 |
| Exact or approximate P value? | Exact |
| P value summary | ns |
| Significantly different (P < 0.05)? | No |
| One- or two-tailed P value? | Two-tailed |
| Sum of ranks in column B, E | 125, 226 |
| Mann-Whitney U | 70 |
| Difference between medians | |
| Median of column B | 697.9, n = 10 |
| Median of column E | 749.5, n = 16 |
| Difference: Actual | −51.61 |
| Difference: Hodges-Lehmann | −44.91 |
| Column H | Arm 8: Pre-treat 5% PKX QID |
| vs. | vs. |
| Column F | Arm 6: 5% PKX QID |
| Mann Whitney test | |
| P value | 0.4291 |
| Exact or approximate P value? | Exact |
| P value summary | ns |
| Significantly different (P < 0.05)? | No |
| One- or two-tailed P value? | Two-tailed |
| Sum of ranks in column F, H | 440, 380 |
| Mann-Whitney U | 170 |
| Difference between medians | |
| Median of column F | 579.0, n = 20 |
| Median of column H | 499.5, n = 20 |
| Difference: Actual | −79.53 |
| Difference: Hodges-Lehmann | −63.35 |

TABLE 16
OGD - Statistics

| Column F | Arm 6: 5% PKX QID |
|---|---|
| vs. | vs. |
| Column D | Arm 4: Pre-treat 5% PKX BID |
| Mann Whitney test | |
| P value | 0.6395 |
| Exact or approximate P value? | Exact |
| P value summary | ns |
| Significantly different (P < 0.05)? | No |
| One- or two-tailed P value? | Two-tailed |
| Sum of ranks in column D, F | 392, 428 |
| Mann-Whitney U | 182 |
| Difference between medians | |
| Median of column D | 565.0, n = 20 |
| Median of column F | 579.0, n = 20 |
| Difference: Actual | 13.98 |
| Difference: Hodges-Lehmann | 19.82 |
| Column G | Arm 7: Pre-treat 2% PKX QID |
| vs. | vs. |
| Column F | Arm 6: 5% PKX QID |
| Mann Whitney test | |
| P value | 0.7616 |
| Exact or approximate P value? | Exact |
| P value summary | ns |
| Significantly different (P < 0.05)? | No |
| One- or two-tailed P value? | Two-tailed |
| Sum of ranks in column F, G | 401, 340 |
| Mann-Whitney U | 169 |
| Difference between medians | |
| Median of column F | 579.0, n = 20 |
| Median of column G | 542.5, n = 18 |
| Difference: Actual | −36.49 |
| Difference: Hodges-Lehmann | −24.74 |

TABLE 17
OGD - Statistics

| Column H | Arm 8: Pre-treat 5% PKX QID |
|---|---|
| vs. | vs. |
| Column G | Arm 7: Pre-treat 2% PKX QID |
| Mann Whitney test | |
| P value | 0.5151 |
| Exact or approximate P value? | Exact |
| P value summary | ns |
| Significantly different (P < 0.05)? | No |
| One- or two-tailed P value? | Two-tailed |
| Sum of ranks in column G, H | 374, 367 |
| Mann-Whitney U | 157 |
| Difference between medians | |
| Median of column G | 542.5, n = 18 |
| Median of column H | 499.5, n = 20 |
| Difference: Actual | −43.04 |
| Difference: Hodges-Lehmann | −37.74 |
| Column H | Arm 8: Pre-treat 5% PKX QID |
| vs. | vs. |
| Column D | Arm 4: Pre-treat 5% PKX BID |
| Mann Whitney test | |
| P value | 0.6395 |
| Exact or approximate P value? | Exact |
| P value summary | ns |
| Significantly different (P < 0.05)? | No |
| One- or two-tailed P value? | Two-tailed |
| Sum of ranks in column D, H | 428, 392 |
| Mann-Whitney U | 182 |
| Difference between medians | |
| Median of column D | 565.0, n = 20 |
| Median of column H | 499.5, n = 20 |
| Difference: Actual | −65.55 |
| Difference: Hodges-Lehmann | −38.38 |

While preferred embodiments have been described above and illustrated in the accompanying drawings, it will be evident to those skilled in the art that modifications may be made without departing from this disclosure. Such modifications are considered as possible variants comprised in the scope of the disclosure.

1. A method for the treatment or prevention of dry eye disease in a subject in need thereof comprising the step of:
a) administering to an eye of said subject an effective amount of a gem-difluorinated C-glycopeptide compound of general formula I, or a pharmaceutically acceptable base, addition salt with an acid, hydrate or solvate of the compound of general formula I: 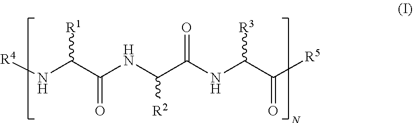
in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain
and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is 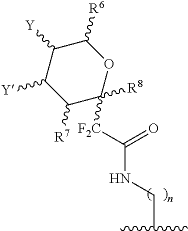
in which:
n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl or acetate group,
R′, R″ independently=H, alkyl, allyl, benzyl, tosylate group, C(═O)-alkyl or C(═O)—Bn,
R′″=H, alkyl, or acetate group,
R⁶ is H, CH₃, CH₂OH, CH₂-glycoside group or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
and
if R¹=R²=H, CH₃, CH₂Ph, CH(CH₃)₂, CH₂CH(CH₃)₂, or CH(CH₃)CH₂CH₃
then R³= 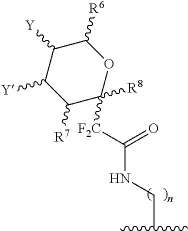
in which: n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R¹=R³=H, CH₃, CH₂Ph, CH(CH₃)₂, CH₂CH(CH₃)₂, or CH(CH₃)CH₂CH₃
then R²= 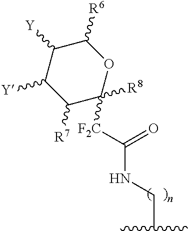
in which: n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²=R³=H, CH₃, CH(CH₃)₂, CH₂CH(CH₃)₂, or CH(CH₃)CH₂CH₃
then R¹= 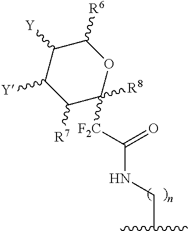
in which: n is an integer between 3 and 4,
Y, Y′ are independent groups
in which Y, Y′ H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

2. The method of any one of claim 1, wherein said subject is a human subject.

3. The method of claim 1, wherein the compound of formula I is a compound of formula II: 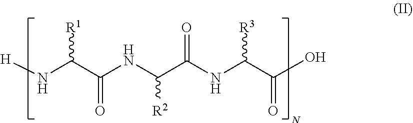
in which: N is an integer between 1 and 5,
and
R¹, R², R³ are independent groups in which two of R¹, R² and R³ are selected from H, CH₃ and the remaining R¹, R² and R³ is 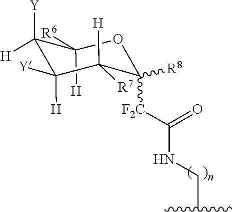
in which: n is an integer between 3 and 4,
Y, Y′ are independent groups
in which Y, Y′=H, OR, N₃, NR′R″ or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is selected from H, CH₃, CH₂OH, CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
R⁸ is a hydrogen atom H or a free or protected alcohol function,
and
if R¹=R²=H or CH₃,
then R³= 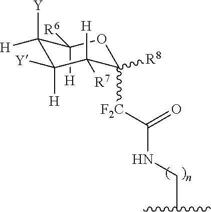
in which: n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is selected from H, CH₃, CH₂OH, CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R¹=R³=H or CH₃,
then R²= 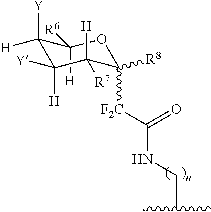
in which: n is an integer between 3 and 4,
Y, Y′ are independent groups
in which Y, Y′=H, OR, N₃, NR′R″, SR′″,
where R=H, benzyl, trimethylsilyl, tert-butyldimethylsilyl, tert-butyldiphenylsilyl, or acetate group,
R′, R″ independently=H, alkyl, allyl,
Bn, tosylate, C(═O)-alkyl, or C(═O)—Bn,
R′″=H, alkyl, or acetate group,
R⁶ is selected from H, CH₃, CH₂OH, or CH₂-OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R²=R³=H or CH₃,
then R¹= 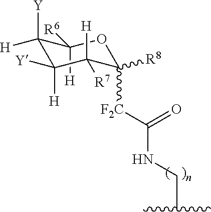
in which: n is an integer between 3 and 4,
Y, Y′ are independent groups
in which Y, Y′ H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)-Bn,
R′″=H, alkyl, or acetate group,
R⁶ is selected from H, CH₃, CH₂OH, or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom or a free or protected alcohol function.

4. The method of claim 1, wherein said compound of formula I is a compound of formula III:

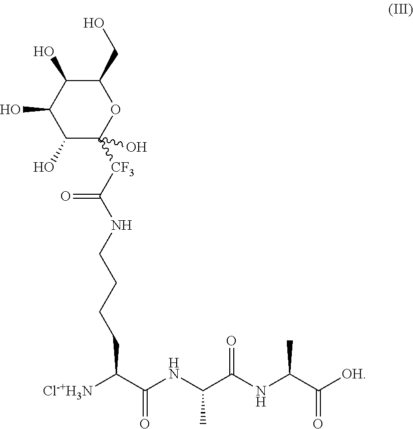

5. The method of claim 1, wherein said effective amount is from about from about 0.01 mg/ml to about 5 mg/ml of said compound of formula I, formula II or formula III.

6. The method of claim 5, wherein said effective amount is with from about 1 mg/ml to about 5 mg/ml of said compound of formula I, formula II or formula III.

7. The method of claim 1, wherein said administration is at least once, twice, three times, or four times per day.

8-28. (canceled)

29. A method for the treatment or prevention of a retinal degenerative disease, an ocular inflammation, or a combination thereof, in a subject in need thereof comprising the step of:
a) administering to an eye of said subject an effective amount of a gem-difluorinated C-glycopeptide compound of general formula I, or a pharmaceutically acceptable base, addition salt with an acid, hydrate or solvate of the compound of general formula I: 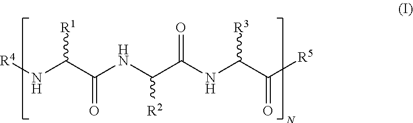
in which:
N is an integer between 1 and 5,
R⁴=H, AA₁, or AA₁-AA₂,
R⁵=OH, AA₁, or AA₁-AA₂,
AA₁ and AA₂ independently represent amino acids with a non-polar side chain
and
R¹, R², R³ are independent groups in which two of R¹, R² and R³ are selected from H, CH₃, CH₂Ph, CH(CH₃)₂, CH₂CH(CH₃)₂ or CH(CH₃)CH₂CH₃ and the remaining R¹, R², R³ is 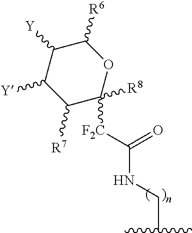
in which:
n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl or acetate group,
R′, R″ independently=H, alkyl, allyl, benzyl, tosylate group, C(═O)-alkyl or C(═O)—Bn,
R′″=H, alkyl, or acetate group,
R⁶ is H, CH₃, CH₂OH, CH₂-glycoside group or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′ or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
and
if R¹=R²=H, CH₃, CH₂Ph, CH(CH₃)₂, CH₂CH(CH₃)₂, or CH(CH₃)CH₂CH₃ then R³= 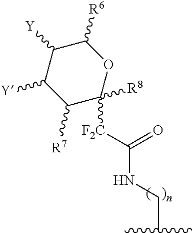
in which: n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tertbutyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R¹=R³=H, CH₃, CH₂Ph, CH(CH₃)₂, CH₂CH(CH₃)₂, or CH(CH₃)CH₂CH₃ then R²= 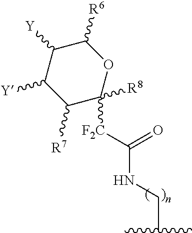
in which: n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function,
if R²=R³=H, CH₃, CH(CH₃)₂, CH₂CH(CH₃)₂, or CH(CH₃)CH₂CH₃
then R¹= 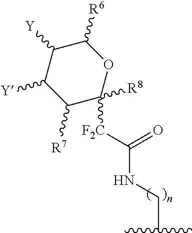
in which: n is an integer between 3 and 4,
Y, Y′ are independent groups
in which Y, Y′ H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is H, CH₃, CH₂OH, CH₂-glycoside group, or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tertbutyldimethylsilyl, tert-butyldiphenylsilyl, or acetate group,
R⁸ is a hydrogen atom H or a free or protected alcohol function.

30. The method of any one of claim 29, wherein said subject is a human subject.

31. The method of claim 29, wherein the compound of formula I is a compound of formula II: 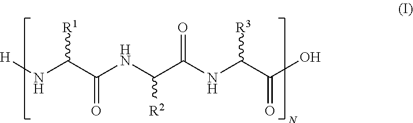
in which: N is an integer between 1 and 5,
and
R¹, R², R³ are independent groups in which two of R¹, R² and R³ are selected from H, CH₃ and the remaining R¹, R² and R³ is 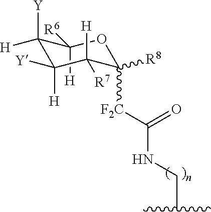
in which: n is an integer between 3 and 4,
Y, Y′ are independent groups
in which Y, Y′=H, OR, N₃, NR′R″ or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is selected from H, CH₃, CH₂OH, CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl or acetate group,
R⁸ is a hydrogen atom H or a free or protected alcohol function,
and
if R¹=R²=H or CH₃,
then R³= 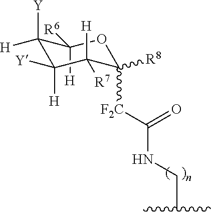
in which: n is an integer between 3 and 4,
Y, Y′ are independent groups
in which Y, Y′=H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)—Bn,
R′″=H, alkyl, or acetate group,
R⁶ is selected from H, CH₃, CH₂OH, CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R¹=R³=H or CH₃,
then R²= 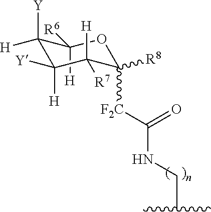
in which: n is an integer between 3 and 4,
Y, Y′ are independent groups
in which Y, Y′=H, OR, N₃, NR′R″, SR′″,
where R=H, benzyl, trimethylsilyl, tert-butyldimethylsilyl, tert-butyldiphenylsilyl, or acetate group,
R′, R″ independently=H, alkyl, allyl,
Bn, tosylate, C(═O)-alkyl, or C(═O)—Bn,
R′″=H, alkyl, or acetate group,
R⁶ is selected from H, CH₃, CH₂OH, or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom H or a free or protected alcohol function,
if R²=R³=H or CH₃,
then R¹= 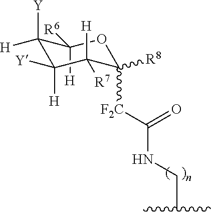
in which: n is an integer between 3 and 4,
Y, Y′ are independent groups
in which Y, Y′ H, OR, N₃, NR′R″, or SR′″,
where R=H, benzyl, trimethylsilyl, tert-butyldimethylsilyl, tert-butyldiphenylsilyl, or acetate group,
R′, R″ independently=H, alkyl, allyl, benzyl, tosylate group, C(═O)-alkyl, or C(═O)-Bn,
R′″=H, alkyl, or acetate group,
R⁶ is selected from H, CH₃, CH₂OH, or CH₂—OGP in which GP is a protector group selected from alkyl, benzyl, trimethylsilyl, tert-butyldimethylsilyl, tert-butyldiphenylsilyl, or acetate group,
R⁷=OH, OGP′, NH₂, N₃, NHGP′, or NGP′GP″ in which GP′ and GP″ are independently selected from alkyl, benzyl, trimethylsilyl, tert-butyldimethylsilyl, tert-butyldiphenylsilyl, or acetate group,
R⁸ is a hydrogen atom or a free or protected alcohol function.

32. The method of claim 29, wherein said compound of formula I is a compound of formula III:

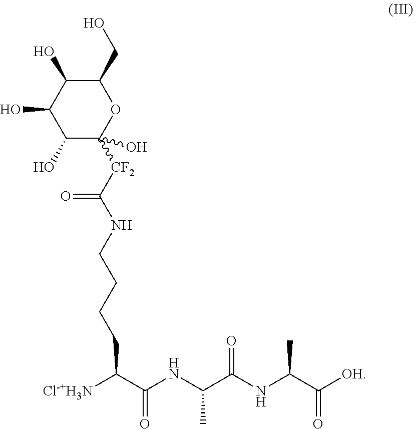

33. The method of claim 29, wherein said effective amount is from about from about 0.01 mg/ml to about 5 mg/ml of said compound of formula I, formula II or formula III.

34. The method of claim 33, wherein said effective amount is with from about 1 mg/ml to about 5 mg/ml of said compound of formula I, formula II or formula III.

35. The method of claim 29, wherein said administration is at least once, twice, three times, or four times per day.

36. The method of claim 29, wherein said retinal degenerative disease is age-related macular degeneration (AMD), diabetic retinopathy, retinitis pigmentosa (RP), retinal vein occlusion, retinal vasculitis, or sarcoidosis.

37-44. (canceled)
